(19) United States
(12) Patent Application Publication
(10) Pub. No.: US 2023/0218727 A1
(43) Pub. Date: Jul. 13, 2023

(54) THROMBIN C-TERMINAL PEPTIDES TO TREAT CORONA VIRAL INFECTIONS

(71) Applicant: In2Cure AB, Lund (SE)

(72) Inventors: Artur Schmidtchen, Lund (SE); Ganna Petruk, Lund (SE); Manoj Puthia, Lund (SE)

(21) Appl. No.: 18/001,611

(22) PCT Filed: Jun. 25, 2021
(86) PCT No.: PCT/EP2021/067450

§ 371 Date: Dec. 13, 2022

(30) Foreign Application Priority Data
Jun. 26, 2020 (EP) 20182667.4

(51) Int. Cl. A61K 38/48; A61P 31/14; G01N 33/569

(52) U.S. Cl. CPC A61K 38/4833; A61P 31/14; G01N 33/56983; G01N 2400/50

(57) ABSTRACT

The present invention relates to TCP peptides for use in methods of treatment of S protein viruses. Notably, said TCP peptides are useful for treatment of inflammation associated with infection by S protein virus. The invention also provides methods for predicting the severity of infection by S protein virus.

### TECHNICAL FIELD

The present invention relates to the field of prevention and/or treatment of viral infections as well as inflammation associated with viral infection. In particular, the invention relates to TCP peptides for use in prevention and/or treatment of inflammation associated with viral infection.

### BACKGROUND

Coronaviruses are a group of enveloped positive-stranded RNA viruses that consist of four structural proteins including spike (S) glycoprotein, envelope (E) protein, membrane (M) protein, and nucleocapsid (N) protein. Spike glycoprotein is the most important surface protein of coronaviruses, such as SARS-CoV-2, which can mediate the entrance to human respiratory epithelial cells by interacting with cell surface receptor angiotensin-converting enzyme 2 (ACE2). COVID-19 disease is associated with a major inflammatory component. Increased cytokine and chemokine production in response to virus infection has been the focus of several recent investigations, and patient morbidity and mortality is mainly caused by the severe systemic inflammation and acute respiratory distress syndrome (ARDS) affecting these patients although differences in ARDS disease phenotypes are noticed.

ARDS is a general systemic inflammatory reaction common for many disease states, such as pneumonia, severe infection, sepsis, burns, or severe trauma. During ARDS, activation of TLRs, such as TLR4 via LPS stimulation, induces an initial systemic pro-inflammatory phase characterized by a massive release of cytokines, acute phase proteins and reactive oxygen species. Additionally, activation of proteolytic cascades, like the coagulation and complement system, takes place in combination with impaired fibrinolysis, and consumption of coagulation factors and other mediators. Clinical symptoms of patients with ARDS therefore in many ways correspond to the pathophysiology seen during severe COVID-19 disease. There is a well-known and established link between high LPS levels in blood and metabolic syndrome (MS), and obesity. Moreover, recent evidence shows that patients with MS are at risk of developing severe COVID-19 disease and ARDS.

Clinical symptoms of patients with COVID-19 are a culmination of complex interactions between the infecting virus and epithelial, blood, and host immune responses. This in turn leads to an induction of local and systemic inflammation. During COVID-19, unresolved infection-inflammation leads to destructive acute disease, which in current treatment attempts is not fully addressed. Current and future antiviral treatments or vaccines only target the invading pathogen, but they cannot interfere with complications caused by a pathologic host response present at epithelial surfaces in the lungs and in blood systemically. Moreover, despite improvements in supportive care and advances in ventilator management, mortality in patients with COVID-19 induced ARDS remains high.

WO2007091959 describes TCP-25 and other peptides for use in treatment of bacterial infections.

### SUMMARY

There is thus an unmet need for methods and compounds useful for treatment of inflammation associated with viral infection by e.g. corona virus.

The present invention surprisingly demonstrate that TCP peptides interfere with the Spike protein (S protein) expressed by a number of viruses and effectively inhibits its proinflammatory actions. Thus, TCP peptides, notably TCP-25 can be utilized for treatment and prevention of the inflammation induced during infection with viruses expressing S protein.

### DESCRIPTION OF DRAWINGS

FIG. 1A-C shows interaction between SARS-CoV-2 protein and LPS in various formulations in vitro using native gel electrophoresis. FIG. 1A shows the migration of S protein alone, or in presence of increasing doses of Escherichia coli LPS. SARS-CoV-2 S protein was incubated with LPS (0-500 μg/ml), separated using Blue Native gel electrophoresis and detected by Western blot. One representative image of three independent experiments is shown (n=3). The marker lane is from the same gel but not transferred to the membrane. It is aligned and included for clarity. Addition of increasing doses of LPS indeed yielded a shift in the migration of S protein, with a reduction of particularly the 400-500 kDa band and increase of high molecular weight material not entering the gel. Under the conditions used, S protein migrated at the molecular mass range of 400-500 kDa. A second higher molecular 700-800 kDa band of less intensity was observed.

FIG. 1B shows Mass spectrometry analysis of the excised protein bands in FIG. 1A. Gel pieces corresponding to the area denoted by the dotted squares on the Western blot were cut out, protein was eluted, digested, and subjected to MALDI mass spectrometry analysis. The results verified that the bands of 400-500 and 700-800 kDa were composed of S protein. S protein was also identified in the high molecular weight fraction found in the samples incubated with LPS. The most intense tryptic fragments obtained from protein S are denoted with the sequence numbers, tryptic peptides from the autodigestion of trypsin are denoted with T. FIG. 1C shows interactions of fluorescence-labeled S protein with E. coli LPS with a KD of 46.7±19.7 nM, using microscale thermophoresis (MST). For control human LPS-receptor CD14 is used, which exhibited a KD of 45.0±24.3 nM to LPS. Mean±S.D. values of six measurements are shown (n=6).

FIG. 2A-C shows the effects of SARS-CoV-2 S protein on LPS-induced responses in THP-1 cells. FIG. 2A shows THP-1-XBlue-CD14 cells treated with increasing concentrations of SARS-CoV-2 S protein (0-10 nM) and a constant dose of LPS (2.5 ng/ml). na; not analyzed. FIG. 2B shows THP-1-XBlue-CD14 cells treated with increasing doses of LPS (0.25-1 ng/ml) and constant amount of S protein (5 nM) (B). na; not analyzed. MTT viability assay for analysis of toxic effects of S protein and LPS on THP-1 cells is shown in lower panels for FIG. 2A and FIG. 2B. FIG. 2C shows cytokine analysis of blood collected from healthy donors, 24 h after treatment with S protein with or without 0.05 and 0.1 ng/ml LPS. Untreated blood was used as a control. The mean±S.E.M. (NF-κB and blood assays) or S.D. (MTT assay) values of four independent experiments performed in duplicate are shown. *, p<0.05; ****, p<0.0001, determined using two-way ANOVA with Sidak's multiple comparisons test (NF-κB and blood assays) or one-way ANOVA with Dunnett's multiple comparison test (MTT assay). FIG. 2D shows a heatmap over cytokines released from peripheral blood mononuclear cells (PBMCs), isolated from three different donors and treated with SARS-CoV-2 S protein (5 nM) and increasing doses of LPS (0.05-0.1 ng/ml) for 8 and 24 h. The cytokines were detected by Luminex multiplex bead assay. Color and values in each box represent mean values of fold increase over untreated cells (n=3).

FIG. 3 shows SARS-CoV-2 S protein combined with LPS boosts inflammation in NF-κB reporter mice. In vivo inflammation imaging in NF-κB reporter mice. LPS alone or in combination with SARS-CoV-2 S protein was subcutaneously deposited on the left and right side, respectively, on the back of transgenic BALB/c Tg(NF-κB-RE-luc)-Xen reporter mice. Non-invasive in vivo bioimaging of NF-κB reporter gene expression was performed using the IVIS Spectrum system. Representative images show bioluminescence at 1, 3 and 6 h after subcutaneous deposition. A bar chart shows measured bioluminescence intensity emitted from these reporter mice. Dotted circles represent area of subcutaneous deposition and region of interest for data analysis. Data are presented as the mean±SEM (n=5 mice for LPS group, 5 mice for LPS and S protein group, 3 mice for buffer control, and 3 mice for S protein control). P values were determined using a one-way ANOVA with Holm-Sidak posttest. **P≤0.01; ***P≤0.001; ****P≤0.0001; NS, not significant.

FIG. 4 shows the anti-inflammatory effects of TCP-25 in THP-1 cells. THP-1-XBlue-CD14 cells were stimulated with 2.5 ng/ml of LPS and 5 nM SARS-CoV-2 S protein and TCP-25 was added at the indicated concentrations. After a 20-24 h incubation period NF-κB activity was evaluated (upper panel). MTT viability assay for analysis of metabolic activity is presented in the lower panels. The mean±S.E.M. (NF-κB assay) or S.D. (MTT assay) values of four independent experiments performed in duplicate are shown. **, p<0.01; ****, p<0.0001, determined using two-way ANOVA with Sidak's multiple comparisons test (NF-κB assay) or one-way ANOVA with Dunnett's multiple comparison test (MTT assay).

FIG. 5 shows the interaction of TCP-25 with SARS-CoV-2 S protein. The binding between TCP-25 and S protein was analyzed by MST following incubation of the peptide at increasing concentrations with 20 nM labeled SARS-CoV-2 S protein. K_{d} constant for TCP-25=3.5±2.5 μM. Mean±S.D. values of six measurements are shown (n=6).

FIG. 6 shows anti-inflammatory effects of TCP-25 and its truncated variants in THP-1 cells. THP-1-XBlue-CD14 cells were stimulated with 2.5 ng/ml of LPS and 5 nM SARS-CoV-2 S protein. TCP-25 or its truncated variants, i.e. GKY20, FYT21 and HVF18, were added at 1 and 5 μM. NF-κB activity was evaluated after a 20-24 h incubation period (upper panel). MTT viability assay for analysis of metabolic activity is presented in the lower panels.

FIG. 7A-B shows the SARS-CoV-2 S protein sequence and endotoxin content FIG. 7A shows an image showing the protein mass of 2019-nCoV full length His-tagged S protein (R683A, R685A), composed of the S protein sequence Val 16-Pro 1213 was produced in HEK293 cells. 1 μg was analyzed on SDS-PAGE followed by staining with Coomassie brilliant blue. The results identified a major band of ˜180-200 kDa. Although the protein has a predicted molecular weight of 134.6 kDa, the result is compatible with the expected mass due to glycosylation. FIG. 7B shows the band was cut off from the gel in former Fig. S1A and analyzed by LC-MS/MS. 110 peptides covered 56% of the SARS-CoV-2 S protein sequence, confirming identity.

FIG. 8A-B shows the interaction specificity of the S protein and the lipid part of the LPS, lipid A. FIG. 8A shows binding of S protein to the lipid part of LPS, lipid A at increasing concentrations (0, 100, 250 and 500 μg/ml). FIG. 8B shows binding of S protein to the lipid part of LPS, lipid A as well as other microbial agonists (LPS_{EC}, LPS_{Pa}, LTA, PGN, Zymosan)

FIG. 9 shows multiple alignment of Spike proteins from viruses belonging to different species. The alignment was performed with Constraint-based Multiple Alignment Tool. Only one strain per specie was included. The sequence coverage of SARS-CoV-2 was set up between 100-50% and identity from 76-30%.

FIG. 10 shows multiple alignment of Spike proteins from viruses belonging to different species. The alignment was performed with Constraint-based Multiple Alignment Tool. The sequence coverage of SARS-CoV-2 was set up between 100-45% and identity from 76-30%.

FIG. 11A-B shows that TCP-25 reduces inflammation caused by S protein and LPS synergism. FIG. 11A shows in vivo inflammation imaging by IVIS in NF-κB reporter mice. LPS alone or with S protein was intratracheally administered in transgenic BALB/c Tg(NF-κB-RE-luc)-Xen reporter mice. For treatment, TCP-25 was also added in the intratracheal administered solution. In vivo bioimaging of NF-κB reporter gene expression was performed using the IVIS Spectrum system. Representative images show bioluminescence at 3 h post intratracheal administration. Bar charts show measured light intensity emitted from these reporter mice at 3, 6, 24 and 48 h post intratracheal administration. P values were determined using a two-way ANOVA. *P<0.05; **P<0.01. FIG. 11B shows cytokine levels in bronchoalveolar lavage fluid from mice 24 h after intratracheally administration. FIG. 11C shows neutrophil numbers in bronchoalveolar lavage fluid from mice 24 h after intratracheal administration. P values were determined using a one-way ANOVA. *P≤0.05; **P≤0.01; ***P≤0.001; ****P≤0.0001.

FIG. 12A-E shows the effects of different doses of SARS-CoV-2 S protein on biophysical properties of LPS. (A and B) Increasing concentrations of LPS (100-500 mg/ml) alone or with S protein (1.48 mM) were incubated for 30 min at 37° C. and then analyzed by DLS (A) and TEM (B). (C) LPS (500 mg/ml) alone or with S protein (5-250 nM) was incubated for 30 min at 37° C. and hydrodynamic radii of the particles in solution were measured by DLS. For DLS, the data are presented as mean±SEM (n=3). P-values were determined using a one-way ANOVA with Sidak's multiple comparisons test. **P≤0.01, ***P≤0.001, ****P≤0.0001. For TEM, one representative picture per each condition is shown (n=3). (D and E) The fluorescence intensity of LPS-FITC (5 mg/ml) alone or with different concentrations of S protein (0.0074-8880 nM) was measured by recording the emission fluorescence spectra between 500 and 600 nm, following excitation at 488 nm. Graphs with spectra are a representative result of three independent experiments (n=3). The change in fluorescence is indicated by an arrow. (E) The fluorescence at 515 nm of FITC-LPS plotted with respect to the concentrations of the protein is presented as floating bars (min to max) with line at median (n=3). A.U., arbitrary units.

FIG. 13 shows a table of diseases involving endotoxins and their links to severe COVID-19. As can be seen in the table, the risk of severe disease in COVID-19 is increased for individuals with metabolic syndrome or COPD.

### DETAILED DESCRIPTION

### Definitions

In this specification, unless otherwise specified, “a” or “an” means “one or more”.

The term ‘amino acid’, as used herein, includes the twenty standard amino acids and their corresponding stereoisomers in the ‘D’ form (as compared to the natural ‘L’ form), omega-amino acids, other naturally-occurring amino acids, unconventional amino acids (e.g., α,α-disubstituted amino acids, N-alkyl amino acids, etc.) and chemically derivatised amino acids (see below).

As used herein the term “approximately” when used in relation to a numerical value refers to +/−10%, preferably +/−5%, more preferably to +/−1%.

The abbreviation “ARDS” as used herein refers to “acute respiratory distress syndrome”. ARDS as used herein is a general systemic inflammatory reaction common for many disease states, such as pneumonia, severe infection, sepsis, burns, or severe trauma. During ARDS, activation of TLRs, such as TLR4 typically via LPS stimulation, induces an initial systemic pro-inflammatory phase characterized by a massive release of cytokines, acute phase proteins and reactive oxygen species. Additionally, activation of proteolytic cascades, like the coagulation and complement system, takes place in combination with impaired fibrinolysis, and consumption of coagulation factors and other mediator. Clinical symptoms of patients with ARDS therefore in many ways correspond to the pathophysiology seen during severe COVID-19 disease.

As used herein the term “chronic obstructive pulmonary disease” (COPD) refers to diseases of the lungs, including chronic bronchitis and emphysema, in which the airways become narrowed, limiting the flow of air to and from the lungs.

The term “Comorbidities” as used herein describes the presence of one or more additional clinical conditions in an individual often co-occurring (that is, concomitant or concurrent with) with a primary clinical condition. Herein the primary clinical condition is typically infection by S protein virus, whereas the comorbidity is any additional clinical condition affecting an individual suffering from such infection by virus.

The term “endotoxin” as used herein refers to components of the exterior cell wall of Gram-negative bacteria that causes adverse effects in humans and animals. Endotoxin can typically be detected by the limulus amebocyte lysate (“LAL”) assay. Preferably, the endotoxin is a lipopolysaccharide (LPS).

The term “Enteritis” as used herein refers to inflammation of the small intestine. It is most commonly caused by food or drink contaminated with pathogenic microbes, but may have other causes such as NSAIDs, cocaine, radiation therapy as well as autoimmune conditions like Crohn's disease or coeliac disease.

The term “Pneumonia” as used herein refers to inflammation of the tissue in the lungs. Pneumonia is usually caused by infection with viruses and/or bacteria and less commonly by other microorganisms, certain medications or conditions such as autoimmune diseases. Pneumonia to be treated according to the present invention is mainly pneumonia induced by infection with S protein virus and potentially co-infection with bacteria.

As used herein the term “S protein virus” refers to a virus comprising the Spike protein (abbreviated as “S protein”) in its protein coat. Notable examples of S protein virus include coronaviruses.

The term “SARS” as used herein refers to Severe acute respiratory syndrome (SARS), which is a viral respiratory illness caused by a coronavirus called SARS-associated coronavirus (SARS-CoV). SARS was first reported in Asia in February 2003. The illness spread to more than two dozen countries in North America, South America, Europe, and Asia before the SARS global outbreak of 2003 was contained.

The term “sequence identity” as used herein refers to the % of identical amino acids or nucleotides between a candidate sequence and a reference sequence following alignment. Thus, a candidate sequence sharing 80% amino acid identity with a reference sequence requires that, following alignment, 80% of the amino acids in the candidate sequence are identical to the corresponding amino acids in the reference sequence. Identity according to the present invention is determined by aid of computer analysis, such as, without limitations, the Clustal Omega computer alignment program for alignment of polypeptide sequences (Sievers et al. (2011 Oct. 11) Molecular Systems Biology 7:539, PMID: 21988835; Li et al. (2015 Apr. 6) Nucleic Acids Research 43 (W1):W580-4 PMID: 25845596; McWilliam et al., (2013 May 13) Nucleic Acids Research 41 (Web Server issue):W597-600 PMID: 23671338, and the default parameters suggested therein. The Clustal Omega software is available from EMBL-EBI at https://www.ebi.ac.uk/Tools/msa/clustalo/. Using this program with its default settings, the mature (bioactive) part of a query and a reference polypeptide are aligned. The number of fully conserved residues are counted and divided by the length of the reference polypeptide. The MUSCLE or MAFFT algorithms may be used for alignment of nucleotide sequences. Sequence identities may be calculated in a similar way as indicated for amino acid sequences. Sequence identity as provided herein is thus calculated over the entire length of the reference sequence.

The term “standard amino acid” refers to any of the twenty genetically-encoded amino acids commonly found in naturally occurring peptides. The standard amino acids are referred to herein both by their IUPAC 1-letter code and 3-letter code. The term “standard amino acid” is used to refer both to free standard amino acids, as well as standard amino acids incorporated into a peptide. For the peptides shown, each encoded amino acid residue, where appropriate, is represented by a single letter designation.

The term “treatment” as used herein refers to any type of treatment or prevention of a disorder, including improvement in the disorder of the subject (e.g., in one or more symptoms), delay in the progression of the disorder, delay the onset of symptoms or slowing the progression of symptoms. Treatment may also be ameliorating or curative treatment. As such, the term “treatment” also includes prophylactic treatment of the individual to prevent the onset of symptoms.

TCP Peptides

The invention relates to compounds comprising thrombin-derived C-terminal (TCP) peptides for use in methods of treatment of inflammation associated with infection by virus containing Spike protein.

As used herein the term “TCP peptide” refers to a peptide comprising or consisting of the amino acid sequence
X₁-X₂-X₃-X₄-X₅-X₆-W-X₈-X₉-X₁₀, wherein
X_{4, 6, 9} is any standard amino acid,
X₁ is I, L or V,
X₂ is any standard amino acid except C,
X₃ is A, E, Q, R or Y,
X₅ is any standard amino acid except R,
X₈ is I or L,
X₁₀ is any standard amino acid except H, wherein said peptide has a length of from 10 to 100, for example from 20 to 100, such as from 18 to 35, for example from 18 to 25 amino acid residues.

In one embodiment, the TCP peptide comprises or consists of the amino acid sequence

X₁-X₂-X₃-X₄-X₅-X₆-W-X₈-X₉-X₁₀-X₁₁-X₁₂-X₁₃, wherein

X_{4, 6, 9, 11} is any standard amino acid,

X₁, is I, L or V

X₂ is any standard amino acid except C

X₃ is A, E, Q, R or Y

X₅ is any standard amino acid except R

X₈ is I or L

X₁₀ is any standard amino acid except H

X₁₂ is I, M or T

X₁₃ is D, K, Q or R

and wherein said peptide has a length of from 20 to 100, such as from 18 to 35, for example from 18 to 25 amino acid residues.

In one embodiment, the TCP peptide comprises or consists of the amino acid sequence

X₁-X₂-X₃-X₄-X₅-X₆-W-X₈-X₉-X₁₀-X₁₁-X₁₂-X₁₃-X₁₄-X₁₅-X₁₆-X₁₇, wherein

X_{4, 6, 9, 11, 14, 15} is any standard amino acid

X₁ is I, L or V

X₂ is any standard amino acid except C

X₃ is A, E, Q, R or Y

X₅ is any standard amino acid except R

X₈ is I or L

X₁₀ is any standard amino acid except H

X₁₂ is I, M or T

X₁₃ is D, K, Q or R

X₁₆ is G or D

X₁₇ is E, L, G, R or K and wherein said peptide has a length of from 20 to 100, such as from 18 to 35, for example from 18 to 25 amino acid residues.

In one embodiment, the TCP peptide has a length of 18 to 35 amino acids, preferably 18-25 amino acids, and comprises or consists of any of the amino acid sequences

(SEQ ID NO: 1)
GKYGFYTHVFRLKKWIQKVIDQFGE,
(SEQ ID NO: 2)
FYTHVFRLKKWIQKVIDQFGE,
(SEQ ID NO: 3)
GKYGFYTHVFRLKKWIQKVI,
(SEQ ID NO: 4)
HVFRLKKWIQKVIDQFGE,
(SEQ ID NO: 5)
KYGFYTHVFRLKKWIQKVIDQFGE
(SEQ ID NO: 6)
GKYGFYTHVFRLKKWIQKVIDQF
(SEQ ID NO: 7)
GKYGFYTHVFRLKKWIQKV.

In one embodiment the TCP peptide has a length of 18 to 35 amino acids, preferably 18-25 amino acids, and comprises or consists of any of the amino acid sequences

(SEQ ID NO: 1)
GKYGFYTHVFRLKKWIQKVIDQFGE,
(SEQ ID NO: 2)
FYTHVFRLKKWIQKVIDQFGE,
(SEQ ID NO: 3)
GKYGFYTHVFRLKKWIQKVI,
or
(SEQ ID NO: 4)
HVFRLKKWIQKVIDQFGE.

In one preferred embodiment the TCP peptide has a length of 18 to 35 amino acids, preferably 18-25 amino acids, and comprises or consists of any of the amino acid sequences

(SEQ ID NO: 1)
GKYGFYTHVFRLKKWIQKVIDQFGE,
or
(SEQ ID NO: 3)
GKYGFYTHVFRLKKWIQKVI.

It is preferred that the TCP peptide is capable of simultaneously binding both to lipopolysaccharides and to the LPS-binding hydrophobic pocket of CD14. Furthermore, it is preferred that the TCP peptide is capable of binding Spike proteins, such as any of the Spike proteins described in the section “Spike protein” herein below.

In one embodiment the TCP peptide is TCP-25. The term “TCP-25” as used herein refers to a peptide consisting of the amino acid sequence GKYGFYTHVFRLKKWIQKVIDQFGE (SEQ ID NO:1). The example section further shows that TCP-25 can be cleaved into the multiple peptides including FYT21, GKY20 and HVF18, i.e. SEQ ID NO:2, 3 and 4. Said peptides also include the amino acid sequences necessary for both lipopolysaccharide (LPS) binding and CD14 binding.

Accordingly, the TCP peptide may in some embodiments comprise or consists of any of these peptides (TCP-25FYT21, GKY20 and HVF18). Preferably the peptide has a length of 18-25 amino acids but, as long as the peptide is based on any of these peptides, the peptide may be up to 35 amino acids long.

In an alternative preferred embodiment the peptide has at least 90% sequence identity with the amino acid sequence GKYGFYTHVFRLKKWIQKVIDQFGE (SEQ ID NO: 1), and preferably the TCP-25 peptide has the amino acid sequence

(SEQ ID NO: 1)
GKYGFYTHVFRLKKWIQKVIDQFGE.

As different amino acids may have side chains providing similar properties, and as the biological effect of a peptide is caused by the side chains of several amino acids cooperating to provide a specific steric structure or chemical/electrical local environment, e.g. by hydrophobic side chains, the peptide may alternatively have at least 90% sequence identity to the TCP-25 sequence. Thus the peptide may correspond to the TCP-25 peptide wherein one or more, up to 1/10^{th}, i.e. up to 3 of the amino acids in the TCP-25 peptide, have been replaced by other amino acids. Despite such replacement, the general activity of the peptide will resemble that of the TCP-25 peptide, (SEQ ID NO:1).

Compound Comprising a TCP Peptide

The invention relates to compounds comprising a TCP peptide for use in treatment of inflammation associated with infection by S protein viruses. Useful TCP peptides are described herein above in the section TCP peptide. In some embodiments said compound may consist of said TCP peptide, e.g. TCP-25. However, in other embodiments, the compound may comprise a TCP peptide conjugated to one or more additional moieties. For example the TCP peptide may comprise one or more amino acids that are modified or derivatised, for example by PEGylation, amidation, esterification, acylation, acetylation and/or alkylation.

For example, the TCP peptide (e.g. TCP-25) may be modified or derivatised as described in international patent application WO2011/036442 on p. 11, I. 1 to p. 15, I. 14.

It is also comprised within the invention that one of more amino acids of the peptide is PEGylated, amidated, acylated, acetylated, alkenylated and/or alkylated. Thus, the peptide may consist of the sequence X₁-X₂-X₃-X₄-X₅-X₆-W-X₈-X₉-X₁₀, wherein each X is as defined above, except that one of more amino acids may be PEGylated, amidated, acylated, acetylated, alkenylated and/or alkylated.

The compound may also be a pharmaceutically acceptable acid or base addition salt of the TCP peptide. The acids which are used to prepare the pharmaceutically acceptable acid addition salts of the TCP peptides are those which form non-toxic acid addition salts, i.e. salts containing pharmacologically acceptable anions, such as the hydrochloride, hydrobromide, hydroiodide, nitrate, sulphate, bisulphate, acid, acetate, lactate, citrate, acid citrate, tartrate, bitartrate, succinate, maleate, fumarate, gluconate, saccharate, benzoate, methanesulphonate, ethanesulphonate, benzenesulphonate, p-toluenesulphonate and pamoate [i.e. 1,1′-methylene-bis-(2-hydroxy-3 naphthoate)] salts, among others.

Virus

The present invention relates to compounds comprising TCP peptides for treatment of infection by S protein virus and for treatment of inflammation associated with infection by virus.

As used herein, “virus” refers to any of a large group of entities referred to as viruses. A virus is a submicroscopic infectious agent that replicates only with the aid of a host. Typically, viruses can only replicate inside the living cells of a host organism. Viruses typically contain a protein coat surrounding an RNA or DNA core of genetic material, and are capable of growth and multiplication only in living cells.

As used herein the term “S protein virus” refers to a virus comprising the Spike protein (abbreviated as “S protein”) in its protein coat. Said S protein may be any of the S proteins described herein below in the section “S protein”.

In one preferred embodiment the S protein virus is a corona virus.

Corona Virus Family

As noted above the invention relates to methods of treating inflammation associated with infection by S protein virus. Preferably, the S protein virus is a corona virus. The coronavirus family, also known as coronaviridae family is a group of enveloped positive-stranded RNA viruses that consist of four structural proteins including spike protein (S protein), envelope (E) protein, membrane (M) protein, and nucleocapsid (N) protein. The Coronaviridae family is of the order Nidovirales. The coronavirus family is divided into four groups; alpha, beta, gamma and delta. The coronavirus according to the invention may be of either group. Corona viruses cause diseases in mammals and birds. In humans, these viruses frequently cause respiratory tract infections of various severity.

In one embodiment, the coronavirus is HCoV-NL63, an alpha coronavirus, associated with upper respiratory tract infections, severe lower respiratory tract infection, and bronchiolitis. A strain of HCoV-NL63 is also known as HCoV-NH (New Haven coronavirus).

In one embodiment, the coronavirus is HCoV-229E, an alpha coronavirus which infects humans and bats and is associated with a range of respiratory symptoms, ranging from the common cold to high-morbidity outcomes such as pneumonia and bronchiolitis.

In one embodiment, the coronavirus is HCoV-HKU1, a beta coronavirus causing acute respiratory syndrome and pneumonia.

In one embodiment, the coronavirus is HCoV-OC43, a beta coronavirus which infects humans and cattle causing acute respiratory infections and pneumonia.

In one embodiment, the coronavirus is SARS-CoV, also known SARS-CoV-1 or SARS (Severe acute respiratory syndrome), a beta coronavirus that causes Severe acute respiratory syndrome

In one embodiment, the coronavirus is MERS-CoV, also known as Middle East respiratory syndrome-related coronavirus, a beta coronavirus that causes Middle East Respiratory Syndrome, or MERS.

In one embodiment, the coronavirus is SARS-CoV 2, which is a newly discovered coronavirus causing the infectious disease COVID-19.

In one embodiment, the coronavirus is PorCov-HKU15, also known as Porcine respiratory coronavirus, PRCV, Swine deltacoronavirus and Porcine deltacoronavirus identified in pigs in the 1980s in Europe. PorCoV-HKU15 affects the respiratory system mainly in pigs.

In one embodiment, the coronavirus is BCoV also known as Bovine coronavirus or BCV, which is a coronavirus which is a member of the species Betacoronavirus causing infection in cattle.

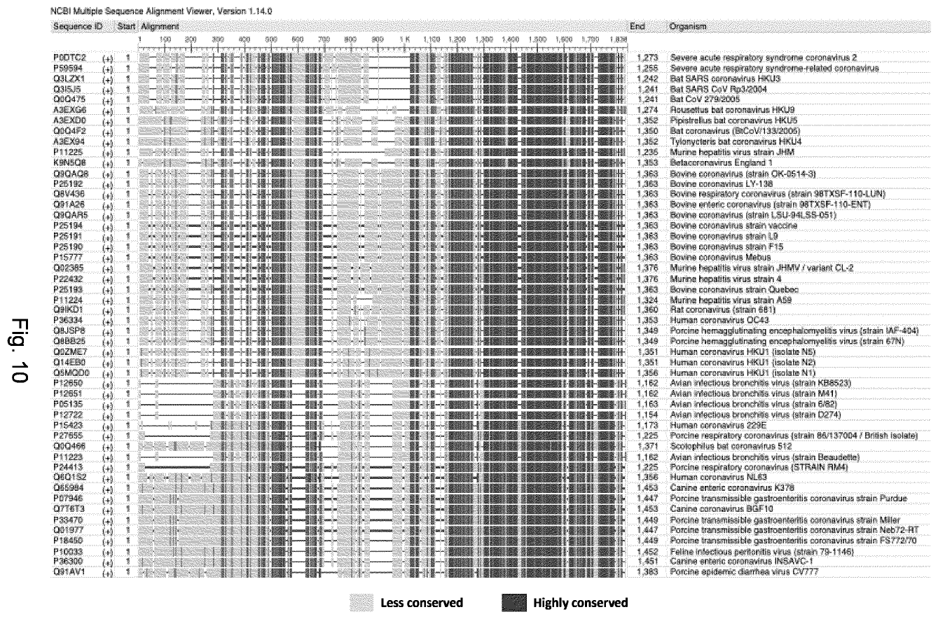

In one embodiment, the coronavirus is any of the viruses mentioned in FIG. 9 and FIG. 10 such as Murine coronavirus (MHV-4) (Murine hepatitis virus), Porcine hemagglutinating encephalomyelitis virus (HEV), Avian infectious bronchitis virus (IBV), Feline coronavirus (FCoV), Porcine epidemic diarrhea virus (PEDV), Tylonycteris bat corona virus; Rat corona virus, Human corona virus, Canine enteric coronavirus, porcine transmissible gastroenteritis coronavirus, feline infectious peritonitis virus, Porcine epidemic diarrhea virus (PEDV).

Spike Protein (S Protein)

S protein viruses described by the invention comprise the Spike protein (S protein) in their protein coat.

The S protein is a large type I transmembrane protein ranging over a size span including proteins of 1,160 amino acids, e.g. avian infectious bronchitis virus (IBV) and proteins of 1,400 amino acids e.g. feline coronavirus (FCoV). The S protein is a glycoprotein, and is thus also known as S glycoprotein or Spike glycoprotein. The terms are used interchangeably herein. S proteins frequently assemble into trimers on the virion surface to form a distinctive “corona”, or crown-like appearance. The ectodomain of all CoV spike proteins share the same organization in two domains: a N-terminal domain named S1 that is responsible for receptor binding (also known as “Spike receptor binding domain”) and a C-terminal S2 domain responsible for fusion.

Spike glycoprotein is the most important surface protein of coronaviruses, such as SARS-CoV-2, and it may mediate the entrance to human respiratory epithelial cells by interacting with cell surface receptor angiotensin-converting enzyme 2 (ACE2).

The S protein according to the present invention may in particular be the S protein of SARS-CoV-2 provided as SEQ ID NO:8 herein and S proteins similar to said protein, such as S proteins sharing at least 20%, such as at least 25% sequence with SEQ ID NO:8.

In one embodiment the S protein may be selected from the group consisting of S proteins of SEQ ID NO:8, SEQ ID NO:9, SEQ ID NO:10, SEQ ID NO:11, SEQ ID NO:12, SEQ ID NO:13 and S proteins sharing at least 20%, such as at least 25%, for example at least 50%, such as at least 70%, for example at least 80%, such as at least 90% sequence identity to one of the aforementioned.

In one embodiment the S protein may be selected from the group consisting of S proteins of SEQ ID NO:8, SEQ ID NO:9, SEQ ID NO:10, SEQ ID NO:11, SEQ ID NO:12, SEQ ID NO:13 and S proteins sharing at least 50% sequence identity to one of the aforementioned.

In one embodiment the S protein may be selected from the group consisting of S proteins of SEQ ID NO:8, SEQ ID NO:9, SEQ ID NO:10, SEQ ID NO:11, SEQ ID NO:12, SEQ ID NO:13 and S proteins sharing 95% sequence identity to one of the aforementioned.

In one embodiment the S protein may be selected from the group consisting of any of the S proteins disclosed in FIG. 9 or 10 or proteins sharing at least 50% sequence identity to any of these.

LPS

The present invention has surprisingly disclosed that the S protein binds to LPS and causes significantly induced inflammation compared to LPS alone.

As used herein, the term “LPS” is an abbreviation for lipopolysaccharide, which is a macromolecule or compound containing lipid and polysaccharide moieties present in the cell walls of mainly in Gram negative bacteria. LPS is preferably proinflammatory LPS.

LPS consists of a lipid A moiety, core polysaccharides and the O-antigen. The lipid A moiety typically consists of fatty acids and disaccharide phosphates, most often a phosphorylated glucosamine disaccharide decorated with multiple fatty acids. The lipid A moiety is a very conserved component of the LPS. The lipid A moiety contributes significantly to LPS' endotoxin activity. In particular, the number of acyl chains on the lipid A moiety may determine its immunostimulatory potential. Hexa-acetylated forms are usually the most immunostimulatory ones, causing the strongest pro-inflammatory immune reactions. For example, penta-acetylated LPS exhibits around 100-fold less immunostimulatory activity than a corresponding hexa-acetylated LPS.

LPS according to the present invention may be any LPS, e.g. an LPS from any Gram negative bacteria. Preferably, the LPS is a proinflammatory LPS, and more preferably, LPS is LPS containing a lipid A moiety, which is hexa-acetylated. For example, the LPS may be an LPS from Escherichia coli or Pseudomonas aeruginosa. In particular, the LPS may be any LPS containing a lipid A moiety which is similar or identical to the lipid A moiety from LPS of Escherichia coli or Pseudomonas aeruginosa. In particular, the LPS may contain any lipid A moiety which has a proinflammatory activity, which is the similar to or higher than the proinflammatory activity of lipid A from LPS of Escherichia coli or Pseudomonas aeruginosa.

Inflammation Associated with Infection by S Protein Virus

The present invention relates to compounds for use in prevention or treatment of inflammation associated with infection by S protein virus. As described herein elsewhere the most severe consequences of infection by S protein viruses frequently are caused by inflammations induced by said infection.

As used herein “inflammation” refers the biological response of tissues to harmful stimuli such as pathogens, damaged cells or irritant. “Inflammation” refers to a protective attempt by an organism to remove an injurious stimulus and initiate the healing processes for the tissue affected by the injurious stimulus.

The inflammation to be treated by the methods according to the present invention may be any undesirable inflammation associated with infection by S protein virus. For example, the inflammation may be an inflammatory disease, which is induced by infection with S protein virus.

Thus, said inflammation may for example be an inflammatory disease selected from the group consisting of acute respiratory distress syndrome (ARDS), severe acute respiratory syndrome (SARS), gastroenteritis and upper and/or lower respiratory tract infections.

Said inflammation may for example be pulmonary inflammation, i.e. pneumonitis or inflammation of the lung tissue. Pulmonary inflammation affects the alveoli, which are small structures of the lung that normally facilitates the passage of oxygen from inhaled air to the bloodstream. During pulmonary inflammation, the exchange of oxygen is impaired.

Pulmonary inflammation may be acute or chronic. It is especially characterized by cough, shortness of breath, fatigue, and fever, and may result in the development of fibrotic scar tissue when chronic or untreated.

There are a number of different causes for pulmonary inflammation, including for example viral infection, bacterial infection, pneumonia, bacterial proteins, interstitial lung disease, sepsis, adverse reaction to medications, smoking, ascariasis, inhalation of certain (hazardous) chemicals, and radiation therapy.

Thus, in some embodiments, the pulmonary inflammation if caused by a viral infection, such as by an S protein virus infection. In some embodiments, the pulmonary inflammation is caused by a bacterial infection, such as by bacterial LPS. In some embodiments, the pulmonary inflammation is caused by a combined bacterial and viral infection, such as by the combined action of S protein and bacterial LPS. Thus, in some embodiments, there is both S protein virus and bacteria comprising LPS in the lungs. As described herein, S protein may potentiate inflammation caused by LPS. For example, pulmonary inflammation induced by LPS may further be potentiated by infection by S protein virus.

Said gastroenteritis may be any form of gastroenteritis. Said upper and/or lower respiratory tract infections may for example be pneumonia.

In particular, when the virus is SARS-CoV-2, the methods of treatment may be methods of treatment of ARDS associated with SARS-CoV-2 infection.

Individual

The present invention relates to a compound for use in methods of treatment of infection by S protein virus, notably methods of treatment of inflammation associated with infection by S protein virus in an individual in need thereof.

In one embodiment, said individual is a human being. In other embodiments, said individual may be an animal, e.g. a domestic animal, such as an animal selected from the group consisting of pig, cattle, cat, poultry, dog and mink.

As described herein protein S may potentiate inflammation caused by LPS. For example, systemic inflammation is induced by lipopolysaccharide (LPS) may further be potentiated by infection by S protein virus. Also local inflammation induced by lipopolysaccharide (LPS) may further be potentiated by infection by S protein virus.

Accordingly, in an individual suffering from increased levels of LPS, an infection by S protein virus may be extremely harmful, because the infection by S protein virus may potentiate a chronic inflammation induced by LPS.

Accordingly, the individual to be treated with the compounds of the invention may in particular be an individual suffering from a disease or syndrome, which directly [or indirectly] causes increased levels of LPS.

In one embodiment, the individual suffers from an S protein virus infection and a bacterial infection. Accordingly, in some embodiments, the individual suffers from an S protein virus infection and a bacterial infection of the lungs.

Said bacterial infection may be an acute or chronic bacterial infection. The bacterial may be infection by any bacteria, however preferably said bacteria are gram negative bacteria. It is preferred that the bacteria are bacteria expressing LPS, and even more preferably the bacterium expresses any of the LPS described herein above in the section “LPS”. Thus, in one embodiment the bacterium expresses pro-inflammatory LPS, such as LPS containing a lipid A moiety, which is hexa-acetylated. The bacteria may in express LPS, which has a pro-inflammatory activity, which is the similar to or higher than the pro-inflammatory activity of LPS of Escherichia coli or Pseudomonas aeruginosa.

In some embodiments, the individual suffers from an S protein virus infection and has an increased level of LPS in bronchoalveolar lavage.

Thus, the individual to be treated may in addition to infection by S protein virus also suffer from one or more comorbidities.

Said comorbidities may in particular be any comorbidity associated with an increased level of endotoxins. For example, the comorbidity may be any clinical condition associated with increased level of LPS, e.g. with a serum level of LPS of at least 30 pg/ml, such as at least 50 pg/ml.

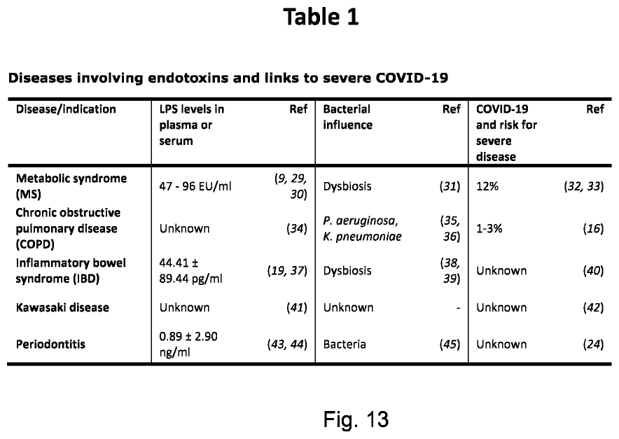

As disclosed herein, individuals suffering from a clinical condition associated with increased levels of LPS, have an increased risk of a severe outcome of infection by S protein virus. For example, the risk of severe disease in COVID-19 is increased for individuals with metabolic syndrome or COPD (see Table 1 in FIG. 13). As described below measurement of LPS levels in COVID-19 patients could have significant diagnostic implications and be of relevance for patient management and treatment decisions. Observations from porcine models also demonstrate that infection with porcine respiratory corona virus, a highly prevalent virus in swine populations significantly sensitizes the lungs to LPS. As shown herein TNF-α levels in human blood is boosted with the combination of LPS and SARS-CoV-2 S protein.

In one embodiment, said individual suffers from metabolic syndrome (MS). The term “metabolic syndrome” refers to a pathological condition in which several metabolic disorders coexist in one single patient. Typically, said disorders include obesity (such as abdominal obesity), insulin resistance, impaired glucose regulation, diabetes mellitus, hypertension, dyslipidemia, microalbunminuria and/or hyperuricemia. MS is associated with high lipopolysaccharide (LPS) levels in blood. MS is a risk factor for developing severe COVID-19 and acute respiratory distress syndrome (ARDS).

In one embodiment, said individual suffers from diabetes. The term “diabetes” include Type 1 diabetes or Type 2 diabetes. Type 1 diabetes is characterized by loss of the insulin-producing beta cells of the islets of Langerhans in the pancreas, leading to a deficiency of insulin. Type 2 diabetes is a chronic disease that is characterised by persistently elevated blood glucose levels (hyperglycemia). Insulin resistance together with impaired insulin secretion from the pancreatic β-cells characterizes the disease. The progression of insulin resistance to type 2 diabetes is marked by the development of hyperglycemia after eating when pancreatic β-cells become unable to produce adequate insulin to maintain normal blood sugar levels (euglycemia).

In one embodiment, the individual suffers from obesity.

In one embodiment, the individual suffers from inflammatory bowel disease. The term “inflammatory bowel disease” or “IBD,” as used herein describes a broad class of diseases characterized by inflammation of at least part of the gastrointestinal tract. IBD symptoms may include inflammation of the intestine and resulting in abdominal cramping and persistent diarrhea. Inflammatory bowel diseases include ulcerative colitis, Crohn's disease, indeterminate colitis, chronic colitis, Collagenous colitis, Lymphocytic colitis, Ischaemic colitis, Diversion colitis, Beliefs disease, discontinuous or patchy disease, ileal inflammation, extracolonic inflammation, granulomatous inflammation in response to ruptured crypts, aphthous ulcers, transmural inflammation, microscopic colitis, diverticulitis and diversion colitis.

In one embodiment, the individual suffers from ulcerative colitis. Ulcerative colitis may occur in people of any age, but most often it starts between ages 15 and 30, or less frequently between ages 50 and 70. Children and adolescents sometimes develop the disease. Ulcerative colitis affects men and women equally and appears to run in some families. Theories about what causes ulcerative colitis abound, but none have been proven. The most popular theory is that the body's immune system reacts to a virus or a bacterium by causing ongoing inflammation in the intestinal wall.

In one embodiment, the individual suffers from Crohn's disease. Crohn's disease is characterized by intestinal inflammation and the development of intestinal stenosis and fistulas; neuropathy often accompanies these symptoms. One hypothesis for the etiology of Crohn's disease is that a failure of the intestinal mucosal barrier, possibly resulting from genetic susceptibilities and environmental factors (e.g., smoking), exposes the immune system to antigens from the intestinal lumen including bacterial and food antigens. Another hypothesis is that persistent intestinal infection by pathogens such as Mycobacterium paratuberculosis, Listeria monocytogenes, abnormal Escherichia coli, or paramyxovirus, stimulates the immune response; or alternatively, symptoms result from a dysregulated immune response to ubiquitous antigens, such as normal intestinal microflora and the metabolites and toxins they produce.

In one embodiment, the individual suffers from or is at risk of acquiring Kawasaki disease. Kawasaki disease, also known as Kawasaki syndrome, also known as mucocutaneous lymph node syndrome is an acute febrile illness of unknown etiology that primarily affects children younger than 5 years of age. It is a form of vasculitis, where blood vessels become inflamed throughout the body. In particular, the compounds of the invention may be used for treatment of a Kawasaki-like disease, also known as PMIS or MIS-C, which is associated with COVID-19.

In one embodiment, the individual suffers from or is at risk of acquiring Paediatric multisystem inflammatory syndrome (PMIS), also known as multisystem inflammatory syndrome in children (MIS-C). PMIS is a systemic disease involving persistent fever, inflammation and organ dysfunction following exposure to SARS-CoV-2, the virus responsible for COVID-19. This syndrome appears similar to Kawasaki disease (a rare disease of unknown origin that affects young children, in which blood vessels become inflamed throughout the body). It can also show features of other serious paediatric inflammatory conditions, including toxic shock and macrophage activation syndromes.

In one embodiment, the individual suffers from chronic obstructive pulmonary disease (COPD). Individuals suffering from COPD may have increased LPS levels derived from bacterial colonization and infection of the lungs. In fact, there is a correlation between LPS levels and bacterial loads during pneumonia.

In one embodiment, the individual suffers from periodontitis, such as from periodontitis caused by infection with Porphyromonas gingivalis and other bacteria can reach the systemic circulation.

In one embodiment the individual is a smoker. Intriguingly, bacterial LPS is an active component of cigarette smoke.

In one embodiment the individual is cattle suffering from or at risk of acquiring shipping fever. In such embodiments the coronavirus may in particular be bovine corona virus.

In one embodiment the individual is a pig, and the coronavirus may porcine respiratory corona virus infection.

In one embodiment the individual is a cat, and the coronavirus may feline CoV.

In one embodiment the individual is poultry, e.g. chicken, and the coronavirus may Infectious bronchitis virus.

Method of Treatment

The invention provides compounds comprising TCP peptides for use methods of treatment.

The compounds comprising TCP peptides may be administered to the individual in need thereof by any suitable route of administration. For example, the compounds may be administered systemically or by local administration directly to the local site affected by the disorder.

In one embodiment, the method of treatment involves parenteral administration

As used herein, “parenteral administration,” means administration through injection or infusion. Parenteral administration includes, but is not limited to, subcutaneous administration, intravenous administration, or intramuscular administration.

In one embodiment, the method of treatment involves nasal administration. By “nasal administration” is meant any form of intranasal administration of any of the constructs of this invention. The constructs may be in an aqueous solution, such as a solution including saline, citrate or other common excipients or preservatives. The constructs may also be in a dry or powder formulation.

In an alternative embodiment, constructs may be administered directly into the lung. “Inhalation administration” or “Intrapulmonary administration” as used herein refers to delivering a drug for absorption to the body in the form of a liquid aerosol mist, solid aerosol particulates or a gaseous substance by inhalation into the lungs.

The skilled person will be able to formulate the compounds of the invention in a manner suitable for the route of administration chosen. For a brief review of present methods useful for drug delivery, see, Langer, Science 249:1527-1533 (1990). Methods for preparing administrable compounds are known or are apparent to those skilled in the art and are described in more detail in, for example, Remington's Pharmaceutical Science, 17th Ed., Mack Publishing Company, Easton, Pa. (1985), and which is hereinafter referred to as “Remington.”

Diagnosis

In one embodiment, the invention provides a method for forecasting the outcome of infection by S protein virus in an individual suffering from or at risk of acquiring infection by said virus, said method comprising the steps of
a. providing a sample from said individual
b. determining endotoxin level in said samples

wherein high endotoxin levels are indicative of a severe outcome.

Said severe outcome may for example be ARDS. Thus, the method may be used for predicting the risk of ARDS, wherein high level of LPS is indicative of risk of ARDS As explained herein above, the present invention discloses that a high level of LPS increases the risk of severe inflammation following infection by S protein viruses.

Accordingly, if an individual has a high level of LPS it may be particularly beneficial to treat said individual with the compound comprising TCP peptides of the invention or to ensure that other means of treatment are available.

The sample may be any sample, e.g. a sample selected from the group consisting of blood, serum, saliva, nasopharyngeal swab samples and bronchoalveolar lavage (BAL) samples.

In some embodiment an LPS level in serum of at least 40 pg/ml, preferably at least 50 pg/ml, for example at least 0.1 ng/ml, such as at least 0.2 ng/ml, for example in the range of 0.1 to 1 ng/ml is considered a “high level”.

In other samples an LPS level of at least 50 pg/ml, for example at least 0.1 ng/ml, such as at least 0.2 ng/ml may considered a “high level”.

The LPS level in the sample may be determined by any suitable method available to the skilled person. For example, the LPS level can be determined by the Limulus amebocyte lysate (LAL) assay, e.g. as described in Example 1 below.

Sequences

TCP Peptide Sequences:

(SEQ ID NO: 1)
GKYGFYTHVFRLKKWIQKVIDQFGE,
(SEQ ID NO: 2)
FYTHVFRLKKWIQKVIDQFGE,
(SEQ ID NO: 3)
GKYGFYTHVFRLKKWIQKVI,
(SEQ ID NO: 4)
HVFRLKKWIQKVIDQFGE,
(SEQ ID NO: 5)
KYGFYTHVFRLKKWIQKVIDQFGE
(SEQ ID NO: 6)
GKYGFYTHVFRLKKWIQKVIDQF
(SEQ ID NO: 7)
GKYGFYTHVFRLKKWIQKV.

SARS-Cov-2 S protein sequence: (QHD43416.1 surface glycoprotein [Severe acute respiratory syndrome coronavirus 2]) >sp|PODTC2|SPIKE_SARS2 Spike glycoprotein OS=Severe acute respiratory syndrome coronavirus 2 OX=2697049 GN=S PE=1 SV=1

(SEQ ID NO: 8)
MFVFLVLLPLVSSQCVNLTTRTQLPPAYTNSFTRGVYYPDKVFRSSVLHS
TQDLFLPFFSNVTWFHAIHVSGTNGTKRFDNPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGY
FKIYSKHTPINLVRDLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASV
YAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYN
YLYRLFRKSNLKPFERDISTEIYQAGSTPCNGVEGFNCYFPLQSYGFQPT
NGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCL
IGAEHVNNSYECDIPIGAGICASYQTQTNSPRRARSVASQSIIAYTMSLG
AENSVAYSNNSIAIPTNFTISVTTEILPVSMTKTSVDCTMYICGDSTECS
NLLLQYGSFCTQLNRALTGIAVEQDKNTQEVFAQVKQIYKTPPIKDFGGF
NFSQILPDPSKPSKRSFIEDLLFNKVTLADAGFIKQYGDCLGDIAARDLI
CAQKFNGLTVLPPLLTDEMIAQYTSALLAGTITSGWTFGAGAALQIPFAM
QMAYRFNGIGVTQNVLYENQKLIANQFNSAIGKIQDSLSSTASALGKLQD
VVNQNAQALNTLVKQLSSNFGAISSVLNDILSRLDKVEAEVQIDRLITGR
LQSLQTYVTQQLIRAAEIRASANLAATKMSECVLGQSKRVDFCGKGYHLM
SFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKE
ELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSC
GSCCKFDEDDSEPVLKGVKLHYT.

SARS-CoV

>tr|Q202E9|Q202E9_CVHSA Spike glycoprotein OS=Human SARS coronavirus OX=694009 GN=S PE=3 SV=1

(SEQ ID NO: 9)
MFIFLLFLTLTSGSDLDRCTTFDDVQAPNYTQHTSSMRGVYYPDEIFRSD
TLYLTQDLFLPFYSNVTGFHTINHTFGNPVIPFKDGIYFAATEKSNVVRG
WVFGSTMNNKSQSVIIINNSTNVVIRACNFELCDNPFFAVSKPMGTQTHT
MIFDNAFNCTFEYISDAFSLDVSEKSGNFKHLREFVFKNKDGFLYVYKGY
QPIDVVRDLPSGFNTLKPIFKLPLGINITNFRAILTAFSPAQDIWGTSAA
AYFVGYLKPTTFMLKYDENGTITDAVDCSQNPLAELKCSVKSFEIDKGIY
QTSNFRVVPSGDVVRFPNITNLCPFGEVFNATKFPSVYAWERKKISNCVA
DYSVLYNSTFFSTFKCYGVSATKLNDLCFSNVYADSFVVKGDDVRQIAPG
QTGVIADYNYKLPDDFMGCVLAWNTRNIDATSTGNYNYKYRYLRHGKLRP
FERDISNVPFSPDGKPCTPPALNCYWPLNDYGFYTTTGIGYQPYRVVVLS
FELLNAPATVCGPKLSTDLIKNQCVNFNFNGLTGTGVLTPSSKRFQPFQQ
FGRDVSDFTDSVRDPKTSEILDISPCSFGGVSVITPGTNASSEVAVLYQD
VNCTDVSTAIHADQLTPAWRIYSTGNNVFQTQAGCLIGAEHVDTSYECDI
PIGAGICASYHTVSLLRSTSQKSIVAYTMSLGADSSIAYSNNTIAIPTNF
SISITTEVMPVSMAKTSVDCNMYICGDSTECANLLLQYGSFCTQLNRALS
GIAAEQDRNTREVFAQVKQMYKTPTLKYFGGFNFSQILPDPLKPTKRSFI
EDLLFNKVTLADAGFMKQYGECLGDINARDLICAQKFNGLTVLPPLLTDD
MIAAYTAALVSGTATAGWTFGAGAALQIPFAMQMAYRFNGIGVTQNVLYE
NQKQIANQFNKAISQIQESLTTTSTALGKLQDVVNQNAQALNTLVKQLSS
NFGAISSVLNDILSRLDKVEAEVQIDRLITGRLQSLQTYVTQQLIRAAEI
RASANLAATKMSECVLGQSKRVDFCGKGYHLMSFPQAAPHGVVFLHVTYV
PSQERNFTTAPAICHEGKAYFPREGVFVFNGTSWFITQRNFFSPQIITTD
NTFVSGNCDVVIGIINNTVYDPLQPELDSFKEELDKYFKNHTSPDVDLGD
ISGINASVVNIQKEIDRLNEVAKNLNESLIDLQELGKYEQYIKWPWYVWL
GFIAGLIAIVMVTILLCCMTSCCSCLKGACSCGSCCKFDEDDSEPVLKGV
KLHYT.

Human coronavirus HKU1 (isolate N5) (HCoV-HKU1)

>sp|QOZME7|SPIKE_CVHN5 Spike glycoprotein OS=Human coronavirus HKU1 (isolate N5) OX=443241 GN=S PE=1 SV=1 MFLIIFILPTTLAVIGDFNCTNSFINDYNKTIPRISEDVVDVSLGLGTYYVLNRVYLNTT

(SEQ ID NO: 10)
MFLIIFILPTTLAVIGDFNCTNSFINDYNKTIPRISEDVVDVSLGLGTYY
VLNRVYLNTTLLFTGYFPKSGANFRDLALKGSIYLSTLWYKPPFLSDFNN
GIFSKVKNTKLYVNNTLYSEFSTIVIGSVFVNTSYTIVVQPHNGILEITA
CQYTMCEYPHTVCKSKGSIRNESWHIDSSEPLCLFKKNFTYNVSADWLYF
HFYQERGVFYAYYADVGMPTTFLFSLYLGTILSHYYVMPLTCNAISSNTD
NETLEYWVTPLSRRQYLLNFDEHGVITNAVDCSSSFLSEIQCKTQSFAPN
TGVYDLSGFTVKPVATVYRRIPNLPDCDIDNWLNNVSVPSPLNWERRIFS
NCNFNLSTLLRLVHVDSFSCNNLDKSKIFGSCFNSITVDKFAIPNRRRDD
LQLGSSGFLQSSNYKIDISSSSCQLYYSLPLVNVTINNFNPSSWNRRYGF
GSFNLSSYDVVYSDHCFSVNSDFCPCADPSVVNSCAKSKPPSAICPAGTK
YRHCDLDTTLYVKNWCRCSCLPDPISTYSPNTCPQKKVVVGIGEHCPGLG
INEEKCGTQLNHSSCFCSPDAFLGWSFDSCISNNRCNIFSNFIFNGINSG
TTCSNDLLYSNTEISTGVCVNYDLYGITGQGIFKEVSAAYYNNWQNLLYD
SNGNIIGFKDFLTNKTYTILPCYSGRVSAAFYQNSSSPALLYRNLKCSYV
LNNISFISQPFYFDSYLGCVLNAVNLTSYSVSSCDLRMGSGFCIDYALPS
SRRKRRGISSPYRFVTFEPFNVSFVNDSVETVGGLFEIQIPTNFTIAGHE
EFIQTSSPKVTIDCSAFVCSNYAACHDLLSEYGTFCDNINSILNEVNDLL
DITQLQVANALMQGVTLSSNLNTNLHSDVDNIDFKSLLGCLGSQCGSSSR
SLLEDLLFNKVKLSDVGFVEAYNNCTGGSEIRDLLCVQSFNGIKVLPPIL
SETQISGYTTAATVAAMFPPWSAAAGVPFSLNVQYRINGLGVTMDVLNKN
QKLIANAFNKALLSIQNGFTATNSALAKIQSVVNANAQALNSLLQQLFNK
FGAISSSLQEILSRLDNLEAQVQIDRLINGRLTALNAYVSQQLSDITLIK
AGASRAIEKVNECVKSQSPRINFCGNGNHILSLVQNAPYGLLFIHFSYKP
TSFKTVLVSPGLCLSGDRGIAPKQGYFIKQNDSWMFTGSSYYYPEPISDK
NVVFMNSCSVNFTKAPFIYLNNSIPNLSDFEAELSLWFKNHTSIAPNLTF
NSHINATFLDLYYEMNVIQESIKSLNSSFINLKEIGTYEMYVKWPWYIWL
LIVILFIIFLMILFFICCCTGCGSACFSKCHNCCDEYGGHNDFVIKASHD
D.

>sp|P15777|SPIKE_CVBM Spike glycoprotein OS=Bovine coronavirus (strain Mebus) OX=11132 GN=S PE=3 SV=1

(SEQ ID NO: 11)
MFLILLISLPMAFAVIGDLKCTTVSINDVDTGAPSISTDIVDVTNGLGTY
YVLDRVYLNTTLLLNGYYPTSGSTYRNMALKGTLLLSRLWFKPPFLSDFI
NGIFAKVKNTKVIKKGVMYSEFPAITIGSTFVNTSYSVVVQPHTTNLDNK
LQGLLEISVCQYTMCEYPHTICHPNLGNKRVELWHWDTGVVSCLYKRNFT
YDVNADYLYFHFYQEGGTFYAYFTDTGVVTKFLFNVYLGTVLSHYYVLPL
TCSSAMTLEYWVTPLTSKQYLLAFNQDGVIFNAVDCKSDFMSEIKCKTLS
IAPSTGVYELNGYTVQPIADVYRRIPNLPDCNIEAWLNDKSVPSPLNWER
KTFSNCNFNMSSLMSFIQADSFTCNNIDAAKIYGMCFSSITIDKFAIPNG
RKVDLQLGNLGYLQSFNYRIDTTATSCQLYYNLPAANVSVSRFNPSTWNR
RFGFTEQFVFKPQPVGVFTHHDVVYAQHCFKAPSNFCPCKLDGSLCVGNG
PGIDAGYKNSGIGTCPAGTNYLTCHNAAQCNCLCTPDPITSKSTGPYKCP
QTKYLVGIGEHCSGLAIKSDYCGGNPCTCQPQAFLGWSVDSCLQGDRCNI
FANFILHDVNSGTTCSTDLQKSNTDIILGVCVNYDLYGITGQGIFVEVNA
TYYNSWQNLLYDSNGNLYGFRDYLTNRTFMIRSCYSGRVSAAFHANSSEP
ALLFRNIKCNYVFNNTLSRQLQPINYFDSYLGCVVNADNSTSSVVQTCDL
TVGSGYCVDYSTKRRSRRAITTGYRFTTFEPFTVNSVNDSLEPVGGLYEI
QIPSEFTIGNMEEFIQTSSPKVTIDCSAFVCGDYAACKSQLVEYGSFCDN
INAILTEVNELLDTTQLQVANSLMNGVTLSTKLKDGVNFNVDDINFSPVL
GCLGSDCNKVSSRSAIEDLLFSKVKLSDVGFVEAYNNCTGGAEIRDLICV
QSYNGIKVLPPLLSVNQISGYTLAATSASLFPPLSAAVGVPFYLNVQYRI
NGIGVTMDVLSQNQKLIANAFNNALDAIQEGFDATNSALVKIQAVVNANA
EALNNLLQQLSNRFGAISSSLQEILSRLDALEAQAQIDRLINGRLTALNV
YVSQQLSDSTLVKFSAAQAMEKVNECVKSQSSRINFCGNGNHIISLVQNA
PYGLYFIHFSYVPTKYVTAKVSPGLCIAGDRGIAPKSGYFVNVNNTWMFT
GSGYYYPEPITGNNVVVMSTCAVNYTKAPDVMLNISTPNLHDFKEELDQW
FKNQTSVAPDLSLDYINVTFLDLQDEMNRLQEAIKVLNQSYINLKDIGTY
EYYVKWPWYVWLLIGFAGVAMLVLLFFICCCTGCGTSCFKICGGCCDDYT
GHQELVIKTSHDD.

MERS-CoV S

>tr|R9UQ53|R9UQ53_MERS Spike glycoprotein OS=Middle East respiratory syndrome-related coronavirus OX=1335626 GN=S PE=1 SV=1

(SEQ ID NO: 12)
MIHSVFLLMFLLTPTESYVDVGPDSVKSACIEVDIQQTFFDKTWPRPIDV
SKADGIIYPQGRTYSNITITYQGLFPYQGDHGDMYVYSAGHATGTTPQKL
FVANYSQDVKQFANGFVVRIGAAANSTGTVIISPSTSATIRKIYPAFMLG
SSVGNFSDGKMGRFFNHTLVLLPDGCGTLLRAFYCILEPRSGNHCPAGNS
YTSFATYHTPATDCSDGNYNRNASLNSFKEYFNLRNCTFMYTYNITEDEI
LEWFGITQTAQGVHLFSSRYVDLYGGNMFQFATLPVYDTIKYYSIIPHSI
RSIQSDRKAWAAFYVYKLQPLTFLLDFSVDGYIRRAIDCGFNDLSQLHCS
YESFDVESGVYSVSSFEAKPSGSVVEQAEGVECDFSPLLSGTPPQVYNFK
RLVFTNCNYNLTKLLSLFSVNDFTCSQISPAAIASNCYSSLILDYFSYPL
SMKSDLSVSSAGPISQFNYKQSFSNPTCLILATVPHNLTTITKPLKYSYI
NKCSRLLSDDRTEVPQLVNANQYSPCVSIVPSTVWEDGDYYRKQLSPLEG
GGWLVASGSTVAMTEQLQMGFGITVQYGTDTNSVCPKLEFANDTKIASQL
GNCVEYSLYGVSGRGVFQNCTAVGVRQQRFVYDAYQNLVGYYSDDGNYYC
LRACVSVPVSVIYDKETKTHATLFGSVACEHISSTMSQYSRSTRSMLKRR
DSTYGPLQTPVGCVLGLVNSSLFVEDCKLPLGQSLCALPDTPSTLTPRSV
RSVPGEMRLASIAFNHPIQVDQLNSSYFKLSIPTNFSFGVTQEYIQTTIQ
KVTVDCKQYVCNGFQKCEQLLREYGQFCSKINQALHGANLRQDDSVRNLF
ASVKSSQSSPIIPGFGGDFNLTLLEPVSISTGSRSARSAIEDLLFDKVTI
ADPGYMQGYDDCMQQGPASARDLICAQYVAGYKVLPPLMDVNMEAAYTSS
LLGSIAGVGWTAGLSSFAAIPFAQSIFYRLNGVGITQQVLSENQKLIANK
FNQALGAMQTGFTTTNEAFRKVQDAVNNNAQALSKLASELSNTFGAISAS
IGDIIQRLDVLEQDAQIDRLINGRLTTLNAFVAQQLVRSESAALSAQLAK
DKVNECVKAQSKRSGFCGQGTHIVSFVVNAPNGLYFMHVGYYPSNHIEVV
SAYGLCDAANPTNCIAPVNGYFIKTNNTRIVDEWSYTGSSFYAPEPITSL
NTKYVAPHVTYQNISTNLPPPLLGNSTGIDFQDELDEFFKNVSTSIPNFG
SLTQINTTLLDLTYEMLSLQQVVKALNESYIDLKELGNYTYYNKWPWYIW
LGFIAGLVALALCVFFILCCTGCGTNCMGKLKCNRCCDRYEEYDLEPHKV
HVH.

Porcine Respiratory Coronovirus

>tr|Q84852|Q84852_9ALPC Spike glycoprotein OS=Porcine respiratory coronavirus OX=11146 GN=S PE=1 SV=1

(SEQ ID NO: 13)
MKKLFVVLVVMPLIYGDKFPTSVVSNCTDQCASYVANVFTTQPGGFIPSD
FSFNNWFLLTNSSTLVSGKLVTKQPLLVNCLWPVPSFEEAASTFCFEGAD
FDQCNGAVLNNTVDVIRFNLNFTTNVQSGKGATVFSLNTTGGVTLEISCY
NDTVSDSSFSSYGEIPFGVTNGPRYCYVLYNGTALKYLGTLPPSVKEIAI
SKWGHFYINGYNFFSTFPIDCISFNLTTGDSDVFWTIAYTSYTEALVQVE
NTAITNVTYCNSYVNNIKCSQLTANLNNGFYPVSSSEVGSVNKSVVLLPS
FLTHTIVNITIGLGMKRSGYGQPIASTLSNITLPMQDNNTDVYCVRSDQF
SVYVHSTCKSALWDNVFKRNCTDVLDATAVIKTGTCPFSFDKLNNYLTFN
KFCLSLSPVGANCKFDVAARTRTNEQVVRSLYVIYEEGDSIVGVPSDNSG
LHDLSVLHLDSCTDYNIYGRTGVGIIRQTNRTLLSGLYYTSLSGDLLGFK
NVSDGVIYSVTPCDVSAQAAVIDGTIVGAITSINSELLGLTHWTITPNFY
YYSIYNYTNDKTRGTPIDSNDVGCEPVITYSNIGVCKNGALVFINVTHSD
GDVQPISTGNVTIPTNFTISVQVEYIQVYTTPVSIDCSRYVCNGNPRCNK
LLTQYVSACQTIEQALAMGARLENMEVDSMLFVSENALKLASVEAFNSSE
TLDPIYTQWPNIGGFWLEGLKYILPSDNSKRKYRSAIEDLLFSKVVTSGL
GTVDEDYKRCTGGYDIADLVCAQYYNGIMVLPGVANADKMTMYTASLAGG
ITLGAFGGGAVSIPFAVAVQARLNYVALQTDVLNKNQQILASAFNQAIGN
ITQSFGKVNDAIHQTSRGLTTVAKALAKVQDVVNTQGQALRHLTVQLQNN
FQAISSSISDIYNRLDELSADAQVDRLITGRLTALNAFVSQTLTRQAEVR
ASRQLAKDKVNECVRSQSQRFGFCGNGTHLFSLANAAPNGMIFFHTVLLP
TAYETVTAWSGICALDGDRTFGLVVKDVQLTLFRNLDDKFYLTPRTMYQP
RVATSSDFVQIEGCDVLFVNTTVSDLPSIIPDYIDINQTVQDILENFRPN
WTVPELTLDVFNATYLNLTGEIDDLEFRSEKLHNTTVELAILIDNINNTL
VNLEWLNRIETYVKWPWYVWLLIGLVVIFCIPLLLFCCCSTGCCGCIGCL
GSCCHSIFSRRQFENYEPIEKVHVH.

Items

The invention may further be defined by the following items:
1. A compound for use in a method of treatment of infection by S protein virus or treatment of inflammation associated with infection by S protein virus in an individual in need thereof, wherein the compound comprises a peptide comprising or consisting of the amino acid sequence
   X₁-X₂-X₃-X₄-X₅-X₆-W-X₈-X₉-X₁₀, wherein
   X_{4, 6, 9} is any standard amino acid,
   X₁ is I, L or V,
   X₂ is any standard amino acid except C,
   X₃ is A, E, Q, R or Y,
   X₅ is any standard amino acid except R,
   X₈ is I or L,
   X₁₀ is any standard amino acid except H,
   wherein said peptide has a length of from 10 to 100 amino acid residues.
2. The compound for use according to item 1, wherein the virus is selected from the group consisting of viruses from the Coronaviridae family.
3. The compound for use according to any one of the preceding items, wherein the virus is selected from the group of viruses disclosed in FIGS. 9 and 10.
4. The compound for use according to any one of the preceding items, wherein the virus is selected from the group consisting of:
   PorCov-HKU15,
   SARS-CoV,
   HCoV NL63
   HKU1,
   MERS-CoV
   SARS-CoV 2, and
   MERS-CoV
5. The compound for use according to any one of the preceding items, wherein the virus is SARS-CoV 2.
6. The compound for use according to any one of the preceding items, wherein the inflammation is a local inflammatory disease associated with said infection by virus.
7. The compound for use according to any one of the preceding items, wherein the inflammation is a pulmonary inflammation.
8. The compound for use according to any one of the preceding items, wherein the inflammation is an inflammatory disease selected from the group consisting of acute respiratory distress syndrome (ARDS), severe acute respiratory syndrome (SARS), gastroenteritis and upper and/or lower respiratory tract infections, for example Pneumonia.
9. The compound for use according to any one of the preceding items, wherein said individual further suffers from a bacterial infection.
10. The compound for use according to any one of the preceding items, wherein said individual further suffers from an acute or chronic bacterial infection, for example infection by gram negative bacteria.
11. The compound for use according to any one of the preceding items, wherein the individual suffers from an S protein virus infection and a bacterial infection of the lungs.
12. The compound for use according to any one of the preceding items, wherein the individual suffers from an S protein virus infection and has an increased level of LPS in bronchoalveolar lavage.
13. The compound for use according to any one of the preceding items, wherein said individual has an increased level of endotoxins.
14. The compound for use according to any one of the preceding items, wherein said individual has increased level of LPS in one or more body fluids.
15. The compound for use according to item 14, wherein said body fluid is selected from the group consisting of blood, serum, saliva, nasopharyngeal swab samples and bronchoalveolar lavage (BAL) samples.
16. The compound for use according to any one of items 12 to 15, wherein said increased level of LPS is a level of at least 50 pg/ml.
17. The compound for use according to any one of items 12 to 16, wherein the LPS is a pro-inflammatory LPS, such as LPS with a hexa-acetylated lipid A moiety.
18. The compound for use according to any one of the preceding items, wherein the individual further suffers from a clinical condition characterised by increased endotoxin levels locally or in blood.
19. The compound for use according to any one of the preceding items, wherein the individual further suffers from one or more clinical conditions selected from the group consisting of COPD, metabolic syndrome, chronic obstructive pulmonary disease, periodontitis, crohns, diabetes and inflammatory bowel disease.
20. The compound for use according to any one of the preceding items, wherein the individual further is a smoker.
21. The compound for use according to any one of the preceding items, wherein the individual is a human being.
22. The compound for use according to any one of the preceding items, wherein the individual is an animal, such as a domestic animal, for example an animal selected from the group consisting of pig, cattle, cat, poultry, dog and mink.
23. The compound for use according to item 22, wherein the compound is for use in treatment of porcine respiratory coronavirus infection, shipping fever induced by bovine coronavirus, feline CoV or infectious bronchitis virus.
24. The compound for use according to any of the preceding items, wherein the compound is formulated for administration by inhalation or for nasal administration or for parenteral administration.
25. The compound for use according to any one of the preceding items, wherein said compound is for use in a method of blocking inflammation induced by Spike protein.
26. The compound for use according to any one of the preceding items, wherein said S protein is selected from the group consisting of SARS CoV-2 S protein of SEQ ID NO: 8 and related S proteins having at least 20%, such as at least 25% sequence identity therewith.
27. The compound for use according to any one of the preceding items, wherein said S protein is selected from the group consisting of S protein of SEQ ID NO: 8, SEQ ID NO: 9, SEQ ID NO: 10, SEQ ID NO: 11, SEQ ID NO: 12, SEQ ID NO: 13 and S proteins sharing 95% sequence identity to one of the aforementioned.
28. The compound for use according to any one of the preceding items, wherein said S protein is selected from the group consisting of S protein of SEQ ID NO: 8, SEQ ID NO: 9, SEQ ID NO: 10, SEQ ID NO: 11, SEQ ID NO: 12, SEQ ID NO: 13 and S proteins sharing 95% sequence identity to one of the aforementioned.
29. The compound for use according to any one of the preceding items, wherein said S protein is any of the S proteins mentioned in FIGS. 9 and 10.
30. The compound for use according to any one of the preceding items, wherein the treatment is preventive treatment.
31. A method for forecasting the outcome of infection by S protein virus in an individual suffering from or at risk of acquiring infection by said virus, said method comprising the steps of
   a. providing a sample from said individual
   b. determining lipopolysaccharide (LPS) level in said samples
   wherein increased LPS levels are indicative of a severe outcome.
32. The method according to item 31, wherein the method is a method for predicting the risk of ARDS, wherein high level of LPS is indicative of risk of ARDS.
33. The method according to any one of items 31 to 32, wherein said sample is selected from the group consisting of blood, serum, saliva, nasopharyngeal swab samples and bronchoalveolar lavage (BAL) samples.
34. The method according to any one of items 31 to 33, wherein said method further comprises administering a compound comprising a peptide as defined in item 1 to said individual if said LPS levels are increased.
35. The method according to any one of items 31 to 34, wherein said increased level of LPS is a level of at least 50 pg/ml.
36. The method according to any one of items 31 to 35, wherein said increased level of LPS is a serum level of LPS of at least 50 pg/ml.
37. The method according to any one of items 31 to 36, wherein said S protein virus is as defined in any one of items 2 to 4.
38. The method according to any one of items 31 to 37, wherein said individual suffers from one or more clinical conditions selected from the group consisting of COPD, metabolic syndrome, chronic obstructive pulmonary disease, periodontitis, crohns, diabetes and inflammatory bowel disease or said individual is a smoker.
39. The compound for use according to any one of the preceding items, wherein the peptide comprises or consists of the amino acid sequence
   X₁-X2-X3-X4-X5-X6-W-X8-X9-X10-X11-X12-X13, wherein
   X4, 6, 9, 11 is any standard amino acid,
   X1, is I, L or V
   X2 is any standard amino acid except C
   X3 is A, E, Q, R or Y
   X5 is any standard amino acid except R
   X8 is I or L
   X10 is any standard amino acid except H
   X12 is I, M or T
   X13 is D, K, Q or R
   and wherein said peptide has a length of from 20 to 100 amino acid residues.
40. The compound for use according to any one of the preceding items, wherein the peptide comprises or consists of the amino acid sequence
   X1-X2-X3-X4-X5-X6-W-X8-X9-X10-X11-X12-X13-X14-X15-X16-X17, wherein
   X4, 6, 9, 11, 14, 15 is any standard amino acid
   X1 is I, L or V
   X2 is any standard amino acid except C
   X3 is A, E, Q, R or Y
   X5 is any standard amino acid except R
   X8 is I or L
   X10 is any standard amino acid except H
   X12 is I, M or T
   X13 is D, K, Q or R
   X16 is G or D
   X17 is E, L, G, R or K
   and wherein said peptide has a length of from 20 to 100 amino acid residues.
41. The compound for use according to any one of the preceding items, wherein the peptide has a length of 18 to 35 amino acids, preferably 18-25 amino acids, and comprises or consists of any of the amino acid sequences

(SEQ ID NO: 1)
GKYGFYTHVFRLKKWIQKVIDQFGE,
(SEQ ID NO: 2)
FYTHVFRLKKWIQKVIDQFGE,
(SEQ ID NO: 3)
GKYGFYTHVFRLKKWIQKVI,
(SEQ ID NO: 4)
HVFRLKKWIQKVIDQFGE,
(SEQ ID NO: 5)
KYGFYTHVFRLKKWIQKVIDQFGE
(SEQ ID NO: 6)
GKYGFYTHVFRLKKWIQKVIDQF
(SEQ ID NO: 7)
GKYGFYTHVFRLKKWIQKV.

42. The compound for use according to any one of the preceding items, wherein the peptide has a length of 18 to 35 amino acids, preferably 18 to 25 amino acids, and comprises or consists of any of the amino acid sequences

(SEQ ID NO: 1)
GKYGFYTHVFRLKKWIQKVIDQFGE,
or
(SEQ ID NO: 3)
GKYGFYTHVFRLKKWIQKVI.

43. The compound for use according to any one of the preceding items, wherein the peptide has at least 90% sequence identity with the amino acid sequence GKYGFYTHVFRLKKWIQKVIDQFGE (SEQ ID NO: 1), preferably the peptide consists of the amino acid sequence GKYGFYTHVFRLKKWIQKVIDQFGE (SEQ ID NO. 1).
44. The compound for use according to any one of the preceding items, wherein one or more of the standard amino acids comprised in the TCP peptide are modified or derivatised.
45. The compound for use according to any one of the preceding items, wherein one or more of the standard amino acids comprised in the TCP peptide are PEGylated, amidated, acylated, acetylated, alkenylated and/or alkylated.

### EXAMPLES

The invention is further illustrated by the following examples, which however should not be construed as limiting for the invention.

### Example 1

This example amongst others define an interaction between S protein and LPS and its link to aggravated inflammation in vitro and in vivo.

Electrophoresis under native conditions demonstrated that S protein binds to Escherichia coli LPS, forming high molecular weight aggregates.

Microscale thermophoresis analysis further defined that the interaction between SARS CoV-2 S protein and Escherichia coli LPS has a Kd of ˜47 nM, similar to the observed affinity between LPS and the human receptor CD14.

Moreover, S protein, when combined with low levels of LPS, boosted nuclear factor-kappa B (NF-κB) and cytokine responses in monocytic THP-1 cells and human blood, respectively.

In an experimental model of localized inflammation, employing NF-κB reporter mice and in vivo bioimaging, S protein in conjunction with LPS significantly increased the inflammatory response when compared with either S or LPS alone.

TCP-25 significantly reduces the inflammation.

As used in the present example the term S protein refers to 2019-nCoV full length His-tagged S protein (R683A, R685A), composed of the S sequence Val 16-Pro 1213.

The term LPS refers to Escherichia coli lipopolysaccharide.

The term TCP-25 refers to a peptide of the following sequence: GKYGFYTHVFRLKKWIQKVIDQFGE (SEQ ID NO:1).

SARS-CoV-2 S Protein Sequence and Endotoxin Content

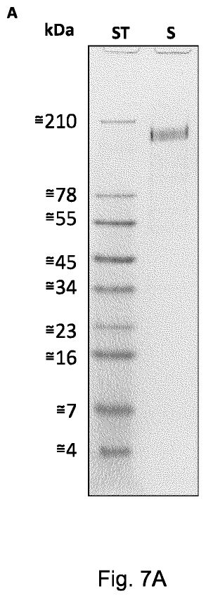

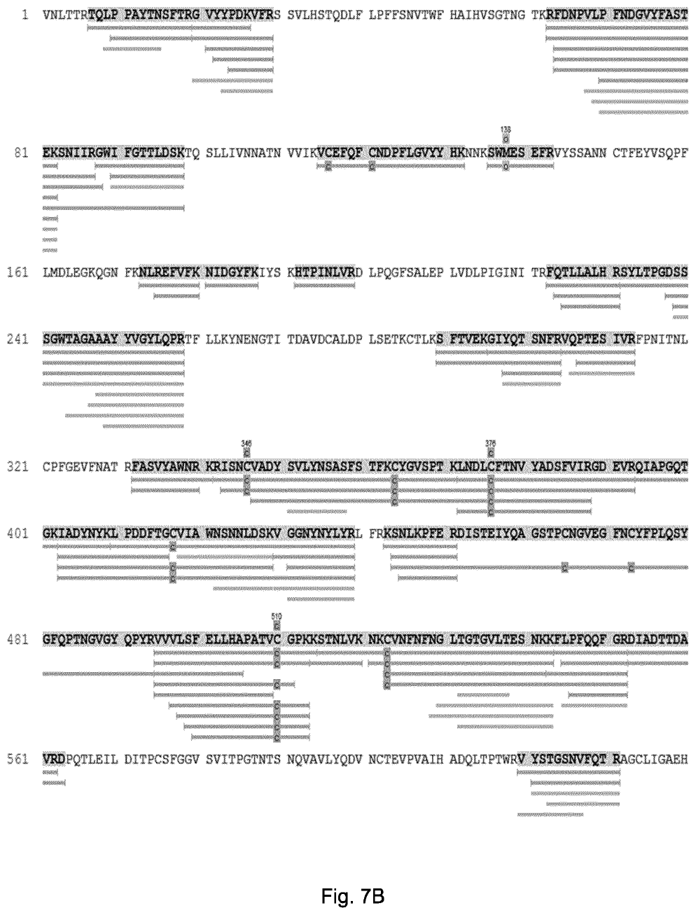

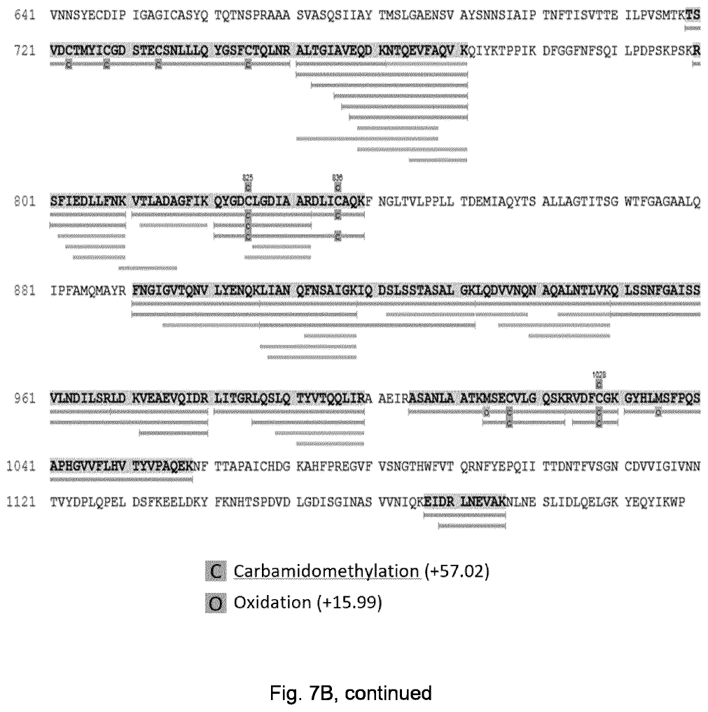

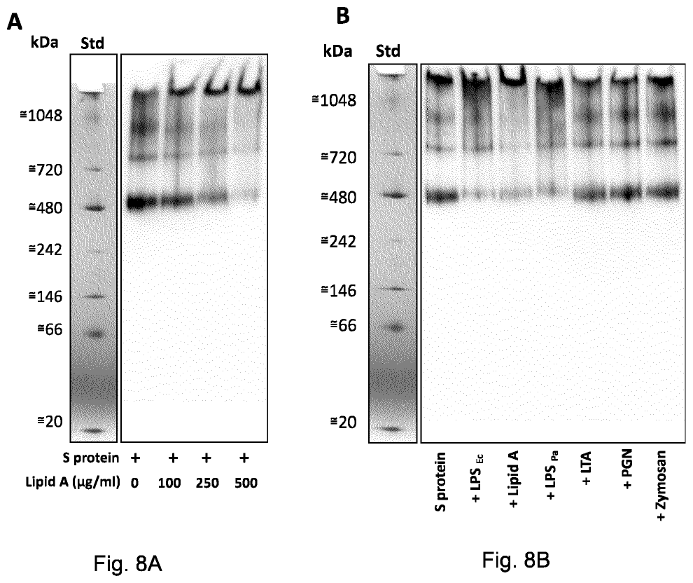

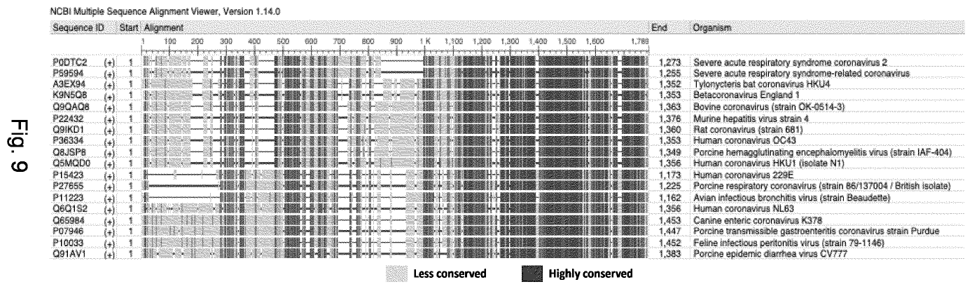

2019-nCoV full length His-tagged S protein (R683A, R685A), composed of the S sequence Val 16-Pro 1213 was produced in HEK293 cells and 1 μg was analyzed on SDS-PAGE followed by staining with Coomassie brilliant blue (FIG. 7A). The results identified a major band of ˜180-200 kDa. Although the protein has a predicted molecular weight of 134.6 kDa, the result is compatible with the expected mass due to glycosylation. Next, the band was cut off from the gel and analyzed by LC-MS/MS (FIG. 7B). 110 peptides covered 56% of the SARS-CoV-2 S protein sequence, confirming identity. Using a limulus amebocyte lysate (LAL) assay, the LPS content in the recombinant S protein was determined to 30 fg/μg protein.

Studies on the Interaction Between SARS-CoV-2 S Protein and LPS

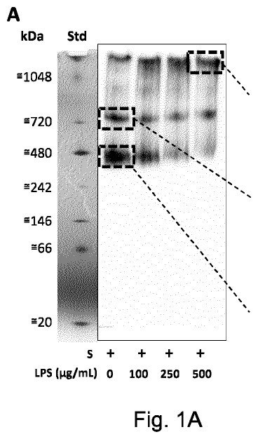

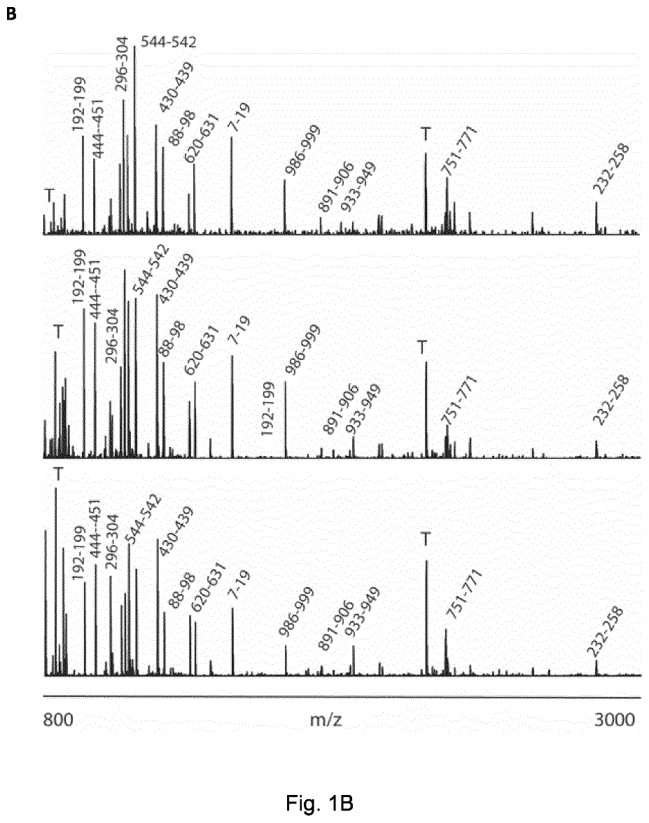

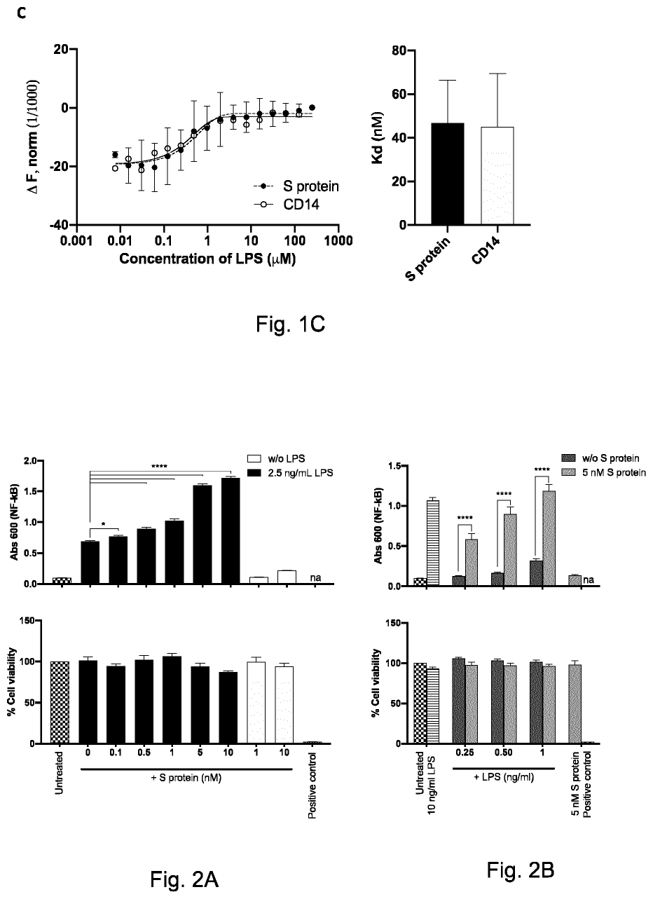

Native gel electrophoresis is used as a tool to assess structural differences in proteins, but also alterations induced by binding to external ligands. The migration of S protein was studied alone, or in presence of increasing doses of Escherichia coli LPS (FIG. 1A). Under the conditions used, S protein migrated at the molecular mass range of 400-500 kDa. A second higher molecular 700-800 kDa band of less intensity was also observed. Addition of increasing doses of LPS indeed yielded a shift in the migration of S protein, with a reduction of particularly the 400-500 kDa band and increase of high molecular weight material not entering the gel. Mass spectrometry of the excised protein bands was then performed. The results verified that the bands of 400-500 and 700-800 kDa were composed of S protein. S protein was also identified in the high molecular weight fraction found in the samples incubated with LPS (FIG. 1B). Analogously, microscale thermophoresis (MST), a highly sensitive technique probing interactions between components in solution, demonstrated interactions of fluorescence-labelled S protein with E. coli LPS, with a K_{D} of 46.7±19.7 nM (FIG. 1C). For control in these experiments human LPS-receptor CD14 was used, which exhibited a K_{D} of 45.0±24.3 nM to LPS. In order to gain more information on the interaction specificity, binding of S protein to the lipid part of LPS, lipid A (FIG. 6A) was evaluated, as well as other microbial agonists (FIG. 6B). S protein was found to interact with lipid A (FIG. 6A) and also LPS from Pseudomonas aeruginosa, whereas not shift in the migration was observed after addition of lipoteichoic acid (LTA), peptidoglycan (PGN), or zymosan. Taken together, using two independent methods probing molecular interactions, a binding of LPS to S was identified. Notably, the affinity of LPS to S protein was in the range of the one observed for LPS binding to the human receptor CD14.

Effects of SARS-CoV-2 S Protein on LPS-Induced Responses In Vitro

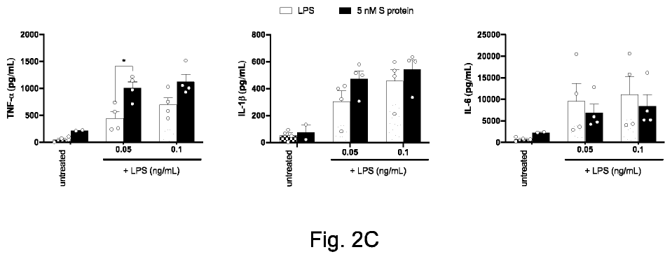

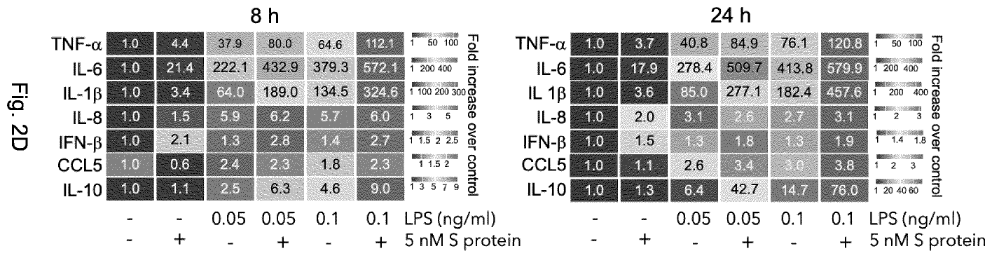

LPS effects depend on specific interactions with components of innate immunity such as LBP, culminating in transfer of lipopolysaccharide from CD14 to Toll-like receptor 4 (TLR4) and its co-receptor MD-2 on the cell surface, leading to activation of downstream inflammatory responses. In order to probe whether the presentation and hence, activity of LPS was altered by the interaction with S protein, the pro-inflammatory effects of S with or without LPS using THP1-XBlue-CD14 cells was studied. After 18-24 h of incubation, NF-κB and AP-1 activation and cell metabolic activity was assessed. In order to assess potential changes in the LPS-response a low dose of LPS of 2.5 ng/ml was used, which is a dose which regularly yields about 20-40% of the maximal response elicited by 100 ng/ml LPS. Addition of S protein at increasing concentrations resulted in a gradual and significant increase in NF-κB/AP-1 activation (FIG. 2A). It was also observed that S protein alone yielded a low, but significant increase in NF-κB/AP-1 activation. Of relevance for the above is that the endogenous levels of LPS in the S preparation were negligible, as they were in the order of 100-1000 lower than the threshold level required for NF-κB activation. In general, patients with a systemic inflammatory response such as seen in sepsis show increased level of the endotoxins in plasma, with levels in the range of 0.1 to 1 ng/ml. In order to mimic those LPS levels, the response of the THP-1 cells to doses ranging from 0.25 ng/ml to 1 ng/ml LPS was determined, with or without the presence of 5 nM of S protein. It was observed that NF-κB activation was significantly boosted even at those low doses of LPS. In particular LPS at 0.25 ng/ml, which alone did not induce a significant increase of NF-κB activation, yielded a significant response together with S protein. It was also observed that LPS at doses of 0.5-1 ng/ml, combined with S protein, yielded response levels produced by 10 ng/ml LPS (FIG. 2B). In these studies, cell viability was regularly measured by the MTT assay, and no significant toxic effects were detected (FIG. 2 A, B). Finally, using human blood, a similar increase of the LPS response was observed. Again, particularly ultra-low levels of LPS, 50 pg/ml, showed boosted TNF-α levels together with S protein (FIG. 2C). Human PBMCs were used for subsequent detailed analyses of inflammatory responses at an early or late time point after stimulation. The results showed that the combination of ultra-low levels of LPS and SARS-CoV-2 S protein yielded significant boosting of TNF-α and interleukin-6 (IL-6) at both time points analysed (8 and 24 h) (FIG. 2D). Both TNF-α and IL-6 are pro-inflammatory cytokines commonly used as markers for inflammation. IL-8 was increased relative to controls, yielding similar levels at both time points, after stimulation with 50 or 100 pg LPS alone. SARS-CoV-2 S protein at 5 nM did neither induce IL-8 alone nor boost the LPS response. Notable was that IFN-β was significantly increased by SARS-CoV-2 S protein alone at both time points, irrespective of the addition of LPS (FIG. 2D). Taken together these results unequivocally demonstrate that S protein increases LPS responses in vitro in monocytic cells and human blood, and in particular, that the activation seen by low, threshold levels of LPS is boosted several-fold by the addition of S protein.

Effects of SARS-CoV-2 S Protein on Endotoxin Responses in an Experimental Mouse Model

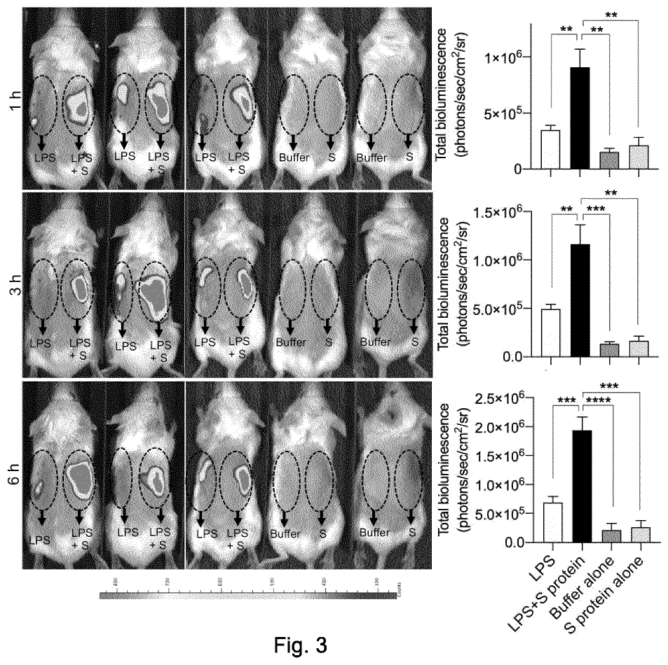

In an experimental animal model, a situation of localized endotoxin induced inflammation was simulated. In previous models, doses of 100 μg LPS injected subcutaneously was utilized, a dose level which yielded a robust and significant LPS response. In this modified model, similarly to the strategy described above on the THP-1 cells, low threshold levels were employed comprising 2 μg LPS which were injected subcutaneously with or without 5 μg S protein. Using mice reporting NF-κB activation, it was found that the addition of S significantly increased the inflammatory response (FIG. 3). S alone at the dose of 5 μg did not yield any significant inflammatory response. Apart from a strongly increased response by the LPS and S combination, it was also observed that the LPS-S mix resulted in a prolonged inflammatory response. Taken together, the results demonstrated that SARS-CoV-2 S protein also retains its boosting effect in conjunction with LPS in a subcutaneous model of endotoxin-driven inflammation.

Effects of TCP-25 on Inflammation Induced by S Protein

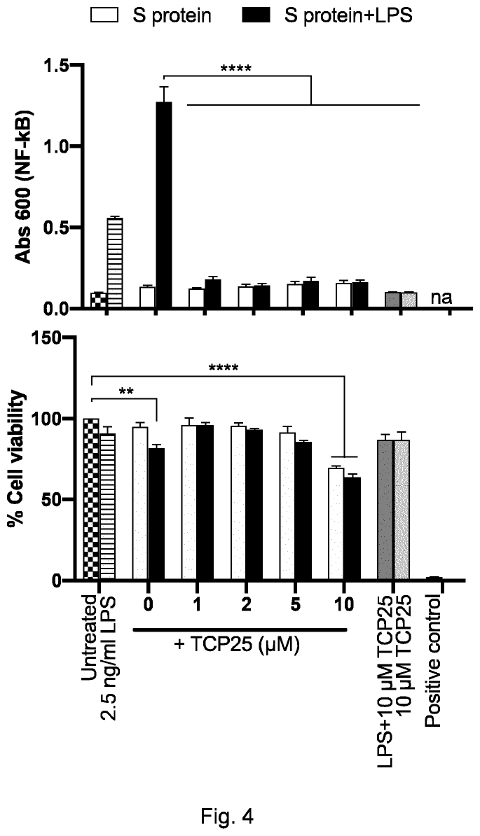

The known endotoxin-blocking effects of TCP-25 depend on specific interactions with both LPS and cells, and thus the structural prerequisites for these anti-inflammatory activities may be separate from those required for an action on the viral S protein. Therefore the activity of the peptide in the presence of S and LPS in vitro using THP1-XBlue-CD14 cells was evaluated. Cells were incubated with S protein (5 nM) with E. coli LPS (2.5 ng/ml) and a dose range of TCP-25 was added. After 18-24 h of incubation, NF-κB and AP-1 activation was assessed. The results showed that TCP-25 abrogated the pro-inflammatory effects of S protein in a background of LPS. (FIG. 4).

Analysis of TCP-25 Binding to SARS-CoV-2 S Protein

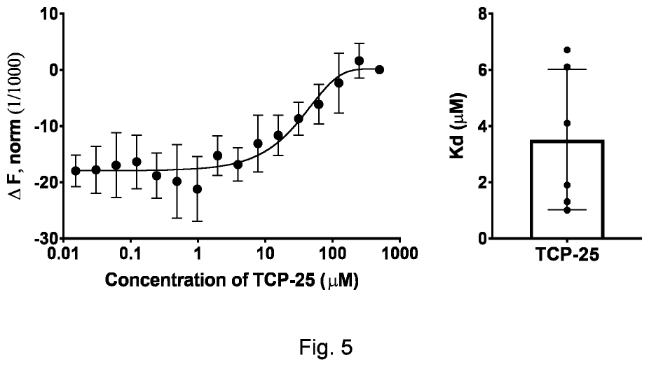

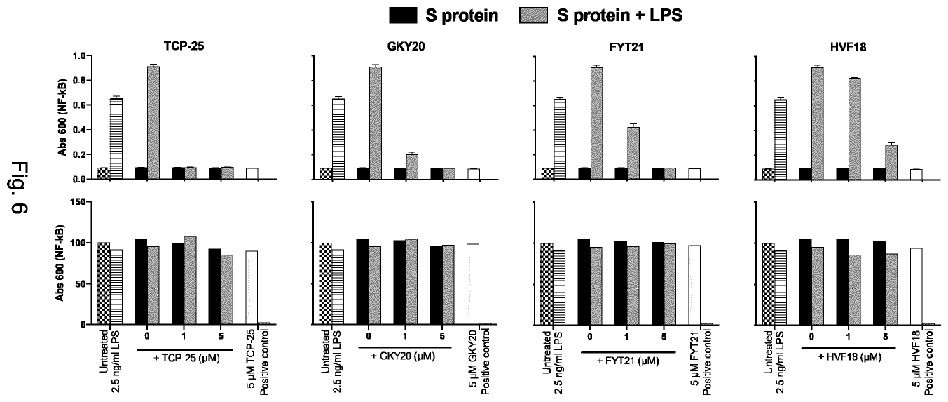

In order to investigate a possible mode of action for TCP-25, it was explored whether the peptide could bind to S protein. Microscale thermophoresis was employed, and the results showed that the K_{D} was 3.5±2.5 μM (FIG. 5). It was observed that the experiments yielded a K_{D} in the range of 1-7 μM, possibly reflecting the complex oligomerization dynamics of TCP-25 and/or S protein in the thermophoresis system. This was not unexpected, as thermophoresis-based results depends on a variety of molecular properties such as size, charge, hydration shell or conformation.

Effects of TCP-25 and Truncated Variants on Inflammation Induced by SARS-CoV-2 S Protein

Previous studies have addressed structure function relationships of TCP-25 and its bioactive epitopes. For example, HVF18 (HVFRLKKWIQKVIDQFGE) is present in wound fluids in vivo. The findings that fragments such as HVF18, and other related truncations of TCP-25 such as FYT21 are present in wound fluids illustrate a concept of redundancy, with multiple bioactive peptide fragments that are simultaneously present. Similar TCP fragments as those that are present in wounds in vivo are generated from synthetically produced TCP-25 (12). TCP-25 cleaved by human neutrophil elastase, a major enzyme that is active during inflammation in blood and organs such as the lungs, generates HVF18 as a major bioactive peptide metabolite. It was also interesting that one of the other major fragments was identical to the previously described GKY20 well as FYT21 (12). From a pharmaceutical perspective it was thus of interest to explore whether these fragments were able to abrogate the proinflammatory effects of S protein in a background of LPS. As shown in FIG. 6, like TCP-25, the peptides GKY20, FYT21, and HVF18 were also able to inhibit SARS-CoV-2 S protein in presence the low dose of LPS used. These data demonstrate that prototypic truncated variants of TCP-25 have a retained activity on S protein induced inflammation.

Effects of TCP-25 on Pulmonary Inflammation Caused by S Protein and LPS Synergism

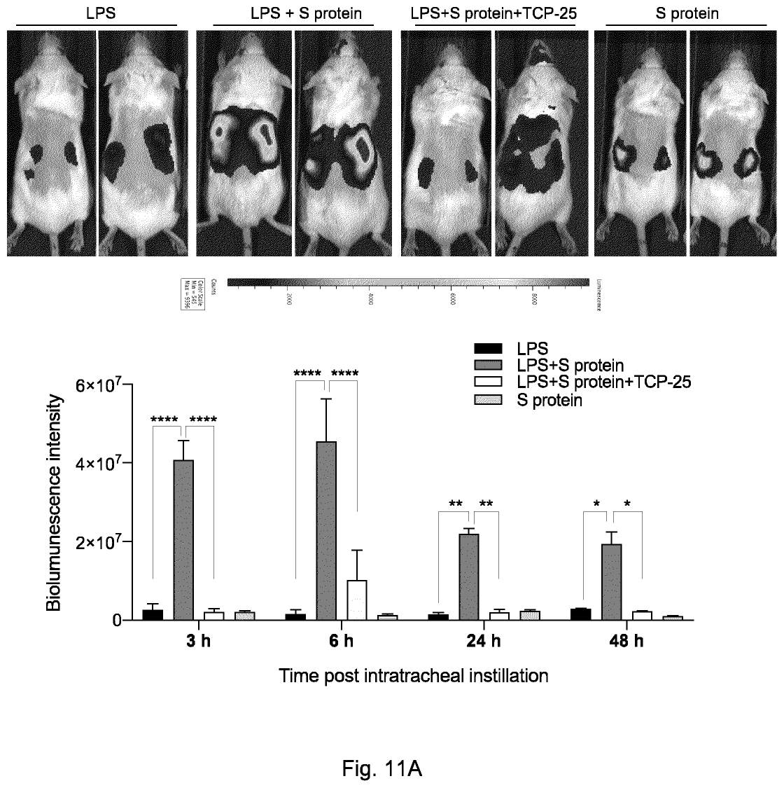

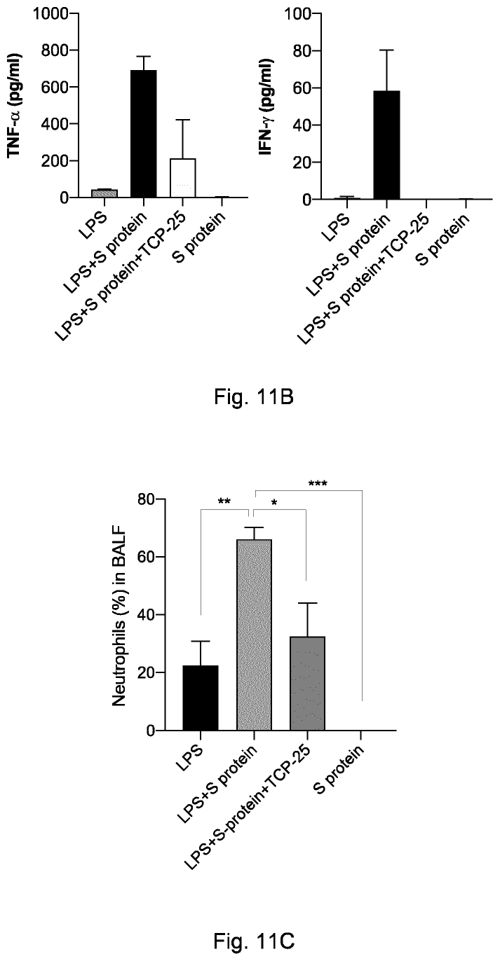

Since TCP-25 was able to abrogate the proinflammatory effects of S protein and LPS (FIG. 4) in vitro, its activity also in a relevant mouse pulmonary inflammation model was evaluated. In this model, similarly to the strategy described above for localized endotoxin induced inflammation model, low, threshold levels were employed comprising LPS (2 μg) or S protein (5 μg) were instilled intratracheally alone, mixed (LPS+S protein) in the presence or the absence of TCP-25 (20 μg). Using mice reporting NF-κB activation, we found that also in this model the addition of S significantly increased the inflammatory response to LPS (FIG. 11A). This was confirmed also by higher release of TNF-α and IFN-γ in bronchoalveolar fluid (FIG. 11B). As well as by increased neutrophil number in bronchoalveolar lavage fluid (FIG. 11C). Interestingly, in the presence of TCP-25 this pro-inflammatory effect of LPS and S combination was completely abrogated (FIGS. 11A, B and C). Taken together, the results demonstrated that SARS-CoV-2 S protein also retains its boosting effect in conjunction with LPS in a pulmonary inflammation model and provide a proof of principle in vivo for the TCP-25 based treatment concept.

Effects of SARS-CoV-2 S Protein on LPS Aggregation

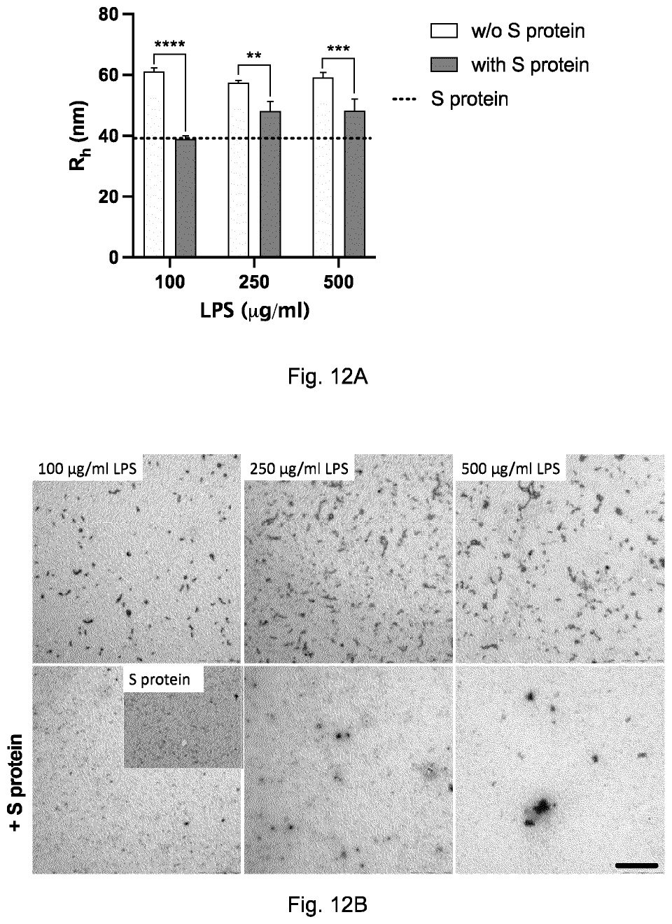

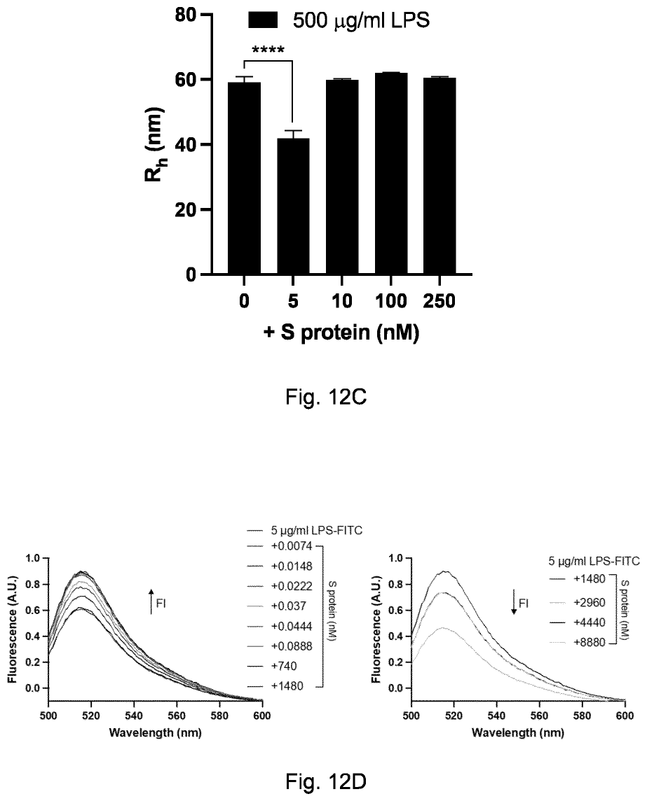

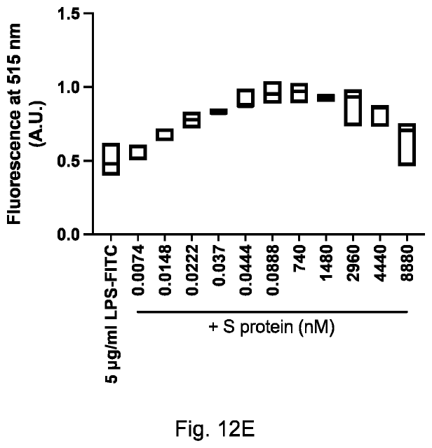

Thus, SARS-CoV-2 S protein both binds to and boosts LPS responses. The interaction and its consequences on the organization of LPS micelles was investigated. Increasing doses of LPS alone or with a constant amount of S protein were incubated and the hydrodynamic radii of the particles in the solution were measured by dynamic light scattering (DLS). The size of LPS particles was found to be 60 nm, and moreover, they were not affected by the concentration of LPS in the dose range studied (FIG. 12A). Incubation of 100 mg/ml of LPS with SARS-CoV-2 S protein, yielded a significant reduction of the hydrodynamic radii of the particles in solution. Notably, the aggregate size was similar to the one observed in the sample with SARS-CoV-2 S protein alone, suggesting a complete dispersion of LPS aggregates by S protein.

A less pronounced, albeit significant, disaggregation was observed when the LPS concentration was increased to 250 and 500 mg/ml, respectively (FIG. 12A). Next, transmission electron microscopy (TEM) was employed in order to further characterize the LPS micelles. Corresponding to the DLS data, a marked disaggregating effect on the LPS micelles was detected using 100 mg/ml LPS (FIG. 12B). In the samples with 250 and 500 mg/ml LPS, the appearance of larger aggregates was noted, suggestive of the LPS-SARS-CoV-2 S protein complexes identified by blue native (BN)-PAGE (FIG. 1A). In order to further study the dose-dependence of the disaggregation and aggregation processes, we incubated 500 mg/ml LPS with increasing doses of S protein (5-250 nM) and analyzed the resulting particles hydrodynamic radii by DLS (FIG. 12C). The results showed that 5 nM of SARS-CoV-2 S protein disaggregated LPS, whereas addition of S protein at higher levels induced aggregation. The data obtained with DLS was further confirmed by studying complexes of fluorescein-labelled LPS (LPS-FITC) in the presence of increasing concentrations of SARS-CoV-2 S protein (FIG. 12D and FIG. 12E). A gradual increase in fluorescence was observed by adding subnanomolar amounts of S protein, indicating a reduction in fluorescein selfquenching due to S protein-induced disaggregation of LPS (FIG. 12D, left panel). With increasing S protein concentrations, the fluorescence level was increased up to a maximum level, indicating a complete dispersion of LPS aggregates. Using higher levels of S protein, however, a decrease in fluorescence intensity of LPS-FITC was noticed, indicating subsequent aggregation (FIG. 12D, right panel). Plotting the fluorescence intensity at 515 nm as function of different concentrations of S protein demonstrated the dose-dependence of the disaggregation and aggregation processes (FIG. 12E). In summary, these data show the dynamic and dose-dependent interactions within SARS-CoV-2 S protein-LPS complexes. Notably, SARSCoV-2 S protein induced a marked disaggregation of LPS at subnanomolar to nanomolar levels.

Discussion

A previously unknown interaction between SARS-CoV-2 S protein and LPS, leading to a boosting of LPS pro-inflammatory actions in vitro as well as in vivo was demonstrated. Moreover, a new therapeutic modality based on treatment with TCP-25 is presented. These results are of clinical importance, as this gives insights in the comorbidities that may increase the risk for severe COVID-19 disease and ARDS, and its pathogenetic steps, as well as provide new therapies based on interference with SARS-CoV-2 S protein's proinflammatory actions.

Materials and Methods

Peptides

The thrombin-derived peptide TCP-25 (GKYGFYTHVFRLKKWIQKVIDQFGE) (SEQ ID NO:1) (97% purity, acetate salt) was synthetized by Ambiopharm (Madrid, Spain). was synthetized by Ambiopharm (Madrid, Spain). GKY20; GKYGFYTHVFRLKKWIQKVI (SEQ ID NO:3), FYT21; FYTHVFRLKKWIQKVIDQFGE (SEQ ID NO:2), and HVF18; HVFRLKKWIQKVIDQFGE (SEQ ID NO:4) were synthesized by Biopeptide (San Diego, Calif., USA). The purity (95%) of the peptides was confirmed by mass spectral analysis (MALDI-TOF, Voyager, Applied Biosystems, Framingham, Mass., USA).

Proteins

SARS-Cov-2 S protein was produced by ACROBiosystems (USA). The sequence contains AA Val 16-Pro 1213 (Accession #QHD43416.1 (R683A, R685A)). Briefly, the 2019-nCoV Full Length S protein (R683A, R685A), His Tag (SPN-C52H4) was expressed in human 293 cells (HEK293) and purified. The protein was lyophilized from a 0.22 μm filtered solution in 50 mM Tris, 150 mM NaCl, pH7.5. Lyophilized product was reconstituted in endotoxin free water, aliquoted and stored at −80° C. according to the manufacturer's protocol. The purity was >85%. Human His-Tag-CD14 (hCD14-his) was produced recombinantly in insect cells by using the Baculovirus Expression Vector System (BEVS). Since this construct is secreted, media was centrifuged in a JLA8-1000 rotor at 8000 g, 20 min, 4° C. and then the supernatant was filtered with a PES 0.45 μm filter top (0.45 μm pore size). Subsequently hCD14-his was purified on a 5 mL HisTrap Excel column (GE Healthcare) by employing AKTA Pure system (GE Healthcare). Eluted fractions were analyzed by precast SDS-PAGE gel (Bio-Rad) stained with BioSafe Coomassie (Bio-Rad) or subjected to Western blot. Peak fractions containing the protein of interest were pooled and digested with tobacco etch virus (TEV) protease to remove the His-Tag. After TEV digestion, the protein solution was run a second time on the His-Trap column. Fractions containing the protein were collected, pooled and purified further on a HiLoad 26/60 Superdex 75 pg gel filtration column. At the end of purification, the purity of hCD14 was estimated to >90%. The protein was aliquoted and stored at −80° C. before use.

Limulus Amebocyte Lysate (LAL) Assay

The content of endotoxin in 1 μg purified SARS-CoV-2 S protein was analyzed using a commercially available Pierce™ Chromogenic Endotoxin Quant Kit (Thermo-Fisher, USA), according to the manufacturer's protocol with small modifications. In particular, the standard curve was done with lipopolysaccharide (LPS) from E. coli (Sigma, USA) in the range between 0.01-10 pg/ml. All samples were prepared in endotoxin-free tubes kept in a thermoblock set to 37° C. At the end of the incubation, 150 μl of each sample were transferred to 96-wells plates and analyzed for absorbance at 410 nm using a spectrophotometer. Pyrogen-free water, used to dissolve the protein, was used as negative control.

SDS-PAGE

1 μg of SARS-Cov-2 S protein was diluted in loading buffer and loaded on 10-20% Novex Tricine pre-cast gel (Invitrogen, USA). The run was performed at 120 V for 1 h. The gel was stained by using Coomassie Brilliant blue (Invitrogen, USA). The image was obtained using a Gel Doc Imager (Bio-Rad Laboratories, USA).

Blue Native (BN)-PAGE

2 μg of SARS-Cov-2 S protein were incubated with 0.1, 0.25 or 0.5 mg/ml of E. coli LPS or Lipid A for 30 min at 37° C. in 20 μL as final volume. At the end of the incubation the samples were separated under native conditions on BN-PAGE (Native PAGE BisTris Gels System 4-16%, Invitrogen) according to the manufacturer's instructions. Proteins were visualized by Coomassie staining. For Western blotting, the material was subsequently transferred to a PVDF membrane using the Trans-Blot Turbo (Bio-Rad, USA). Primary antibodies against the His-tag (1:2000, Invitrogen) were followed by secondary HRP conjugated antibody (1:2000, Dako, Denmark), for detection of S protein. The protein was visualized by incubating the membrane with SuperSignal West Pico Chemiluminescent Substrate (Thermo Scientific, Denmark) for 5 min followed by detection using a ChemiDoc XRS Imager (Bio-Rad). In another set of experiments, 2 μg of SARS-Cov-2 S protein were incubated with 0.25 mg/ml of LPS and Lipid A from E. coli, LPS from P. aeruginosa, LTA and PGN from S. aureus, and zymosan from S. cerevisiae. BN-PAGE and Western blotting were performed as described above. LPS from E. coli and P. aeruginosa as well as Lipid A were purchased from Sigma-Aldrich, whereas LTA, PGN and zymosan were purchased from InvivoGen.

Mass Spectrometry Analysis

After separation by SDS- or BN-PAGE and Coomassie staining, bands in the gels were cut out and the digestion was performed according to Shevchenko et al. (20). Briefly the gel pieces were washed with water, then with a mix of 50 mM ammonium carbonate in 50% acetonitrile (ACN). Gel pieces were shrunk with 100% of acetonitrile and then reduced with 10 mM DTT 30 min 56° C. Alkylation was performed with 55 mM idoacetamide at RT. 10 ng/L of trypsin solution was added to cover the gel-pieces placed on ice and after 1 hour the samples were placed at 37° C. and overnight digestion was performed. The supernatant was acidified using 5% of formic acid and then analyzed by MALDI MS or LC-MS/MS.

MALDI Mass Spectrometry Analysis

For MALDI mass spectrometry analysis, digested SARS-CoV-2 S protein samples were mixed with a solution of 0.5 mg/ml α-Cyano-4-hydroxycinnamic acid (CHCA) 50% ACN/0.1% phosphoric acid solution directly on a stainless MALDI target plate. Subsequent MS analysis was performed on a MALDI LTQ Orbitrap XL mass spectrometer (ThermoScientific, Bremen, Germany). Full mass spectra were obtained by using the FT analyser (Orbitrap) at 60,000 resolution (at m/z 400). Recording of Mass spectra was performed in positive mode with a 800-4000 Da mass range. The nitrogen laser was operated at 27 μJ with automatic gain control (AGC) off mode using 10 laser shots per position. Evaluation of the spectra was performed with Xcalibur v 2.0.7. software (from Thermo Fisher Scientific, San Jos6, CA).

LC-MS/MS

The LC-MS/MS detection was performed on HFX orbitrap equipped with a Nanospray Flex ion source and coupled with an Ultimate 3000 pump (Thermo Fischer Scientific). Peptides were concentrated on an Acclaim PepMap 100 C18 precolumn (75 μm×2 cm, Thermo Scientific, Waltham, Mass.) and then were separated on an Acclaim PepMap RSLC column (75 μm×25 cm, nanoViper, C18, 2 μm, 100 Å) with heating at 45° C. for both the columns. Solvent A (0.1% formic acid) and solvent B (0.1% formic acid in 80% ACN) were used to create a nonlinear gradient to elute the peptides. For the gradient, the percentage of solvent B increased from 4% to 10% in 20 min, increased to 30% in 18 min and then increased to 90% in 2 min and kept it for a further 8 min to wash the columns.

The Orbitrap HFX instrument was operated in data-dependent acquisition (DDA) mode. The peptides were introduced into mass spectrometer via stainless steel Nano-bore emitter (OD 150 μm, ID 30 μm) with the spray voltage of 1.9 kV and the capillary temperature was set 275° C. Full MS survey scans from m/z 350-1600 with a resolution 1200,000 were performed in the Orbitrap detector. The automatic gain control (AGC) target was set to 3×10⁶ with an injecting time of 20 ms. The most intense ions (up to 20) with charge state 2-5 from the full scan MS were selected for fragmentation in Orbitrap. MS2 precursors were isolated with a quadrupole mass filter set to a width of 1.2 m/z. Precursors were fragmented by collision-induced dissociation (CID) and detected in ion trap detector with rapid scan rate. The collision energy for CID was 27%. The resolution was set at 15000 and the values for the AGC target and inject time were 2×10³ and 60 ms, respectively for MS/MS scans. The duration of dynamic exclusion was set 15 s and the mass tolerance window 10 ppm. MS/MS data was acquired in centroid mode. MS/MS spectra were searched with PEAKS (version 10) against UniProt Homo Sapiens (version 2020_02). A 10 ppm precursor tolerance and 0.02 Da fragment tolerance were used as the MS settings. Trypsin was selected as enzyme with one missed cleavage allowance, methionine oxidation and deamidation of aspargine and glutamine were treated as dynamic modification and carbamidomethylation of cysteine as a fixed modification. Maximum of post-translational modifications (PTM) per peptide was 2.

Microscale Thermophoresis

Microscale thermophoresis (MST) was performed on a NanoTemper Monolith NT.115 apparatus (Nano Temper Technologies, Germany). 40 μg SARS-CoV-2 S protein and 100 μg of recombinant hCD14 were labeled by Monolith NT Protein labelling kit RED—NHS (Nano Temper Technologies, Germany) according to the manufacturer's protocol. 5 μl of 20 nM labeled SARS-CoV-2 S protein or 20 nM labeled hCD14 were incubated with 5 μl of increasing concentrations of LPS (250-0.007 μM) in 10 mM Tris pH 7.4. Then, samples were loaded into standard glass capillaries (Monolith NT Capillaries, Nano Temper Technologies) and the MST analysis was performed (settings for the light-emitting diode and infrared laser were 80%). Results shown are mean values±SD of six measurements. The binding between SARS-CoV-2 S protein and TCP-25 was assessed by MST following the incubation of S protein with increasing concentration of TCP-25 in 10 mM Tris pH 7.4. Analysis was performed as described above.

NF-κB Activation Assay

THP1-XBlue-CD14 reporter cells (InvivoGen, San Diego, USA) were seeded in 96 well plates in phenol red RPMI, supplemented with 10% (v/v) heat-inactivated FBS and 1% (v/v) Antibiotic-Antimycotic solution (180,000 cells/well). Cells were treated with 2.5 ng/ml LPS (Sigma, USA) with increasing concentrations (0.1-10 nM) of SARS-CoV-2 S protein or with 5 nM SARS-CoV-2 S protein with increasing concentrations of LPS (0.25-1 ng/ml). Then, the cells were incubated at 37° C. for 20 h. At the end of incubation, the NF-κB activation was analyzed according to the manufacturer's instructions (InvivoGen, San Diego, USA), i.e. by mixing 20 μl of supernatant with 180 μl of SEAP detection reagent (Quanti-Blue™, InvivoGen), followed by absorbance measurement at 600 nm. Data shown are mean values±SEM obtained from at least four independent experiments all performed in triplicate. For evaluation of the effects of TCP-25 on the NF-κB activation induced by S and LPS, increasing doses of the peptide were added to THP1-XBlue-CD14 cells in combination with 2.5 μg/mL of LPS and 5 nM of S protein. The NF-κB activation was detected as described above. Anti-inflammatory effects of TCP-25 and its truncated versions were studied by stimulating THP1-XBlue-CD14 cells with 5 nM S protein and 2.5 μg/ml LPS, with addition of 1 and 5 μM of TCP-25, GKY20, FYT21 or HVF18, respectively. NF-κB activation was detected as described above.

MTT Assay

The cytotoxicity of the treatments was evaluated by adding 0.5 mM Thiazolyl Blue Tetrazolium Bromide to the cells remaining from NF-κB activation assay. After 2 h of incubation at 37° C., cells were centrifuged at 1000 g for 5 min and then the medium was removed. Subsequently, the formazan salts were solubilized with 100 μL of 100% DMSO (Duchefa Biochemie, Haarlem). Absorbance was measured at a wavelength of 550 nm. Cell survival was expressed as percentage of viable cells in the presence of different treatment compared with untreated cells. Lysed cells were used as positive control. Data shown are mean values±SD obtained from at least four independent experiments all performed in triplicate.

Blood Assay

Fresh venous blood was collected in the presence of lepirudin (50 mg/ml) from healthy donors. The blood was diluted 1:4 in RPMI-1640-GlutaMAX-1 (Gibco) and 1 mL of this solution was transferred to 24-well plates and stimulated with 0.05 or 0.1 ng/mL of LPS in the presence or the absence of 5 nM of SARS-CoV-2 S protein. After 24 h incubation at 37° C. in 5% CO₂, the plate was centrifuged for 5 min at 1000 g and then the supernatants were collected and stored at −80° C. before analysis. The experiment was performed at least 4 times by using blood from different donors each time.

ELISA

The cytokines tumor necrosis factor alpha (TNF-α), interleukin-1p (IL-1p) and interleukin-6 (IL-6), were measured in human plasma obtained after the blood experiment described above. The assay was performed by using human inflammation DuoSet® ELISA Kit (R&D Systems) specific for each cytokine, according to the manufacturer's instructions. Absorbance was measured at a wavelength of 450 nm. Data shown are mean values±SEM obtained from at least four independent experiments all performed in duplicate.

Mouse Inflammation Model

The immunomodulatory effects of 5 μg SARS-CoV-2 S protein in combination or not with 2 μg LPS/mouse were tested employing BALB/c tg (NF-κB-RE-Luc)-Xen reporter mice (Taconic Biosciences, Albany, N.Y., USA, 10-12 weeks old). The mice carry a transgene containing 6 NFκB-responsive elements (RE) from the CMVα (immediate early) promoter placed upstream of a basal SV40 promoter, and a modified firefly luciferase cDNA (Promega pGL3). The reporter is activated under conditions where NFκB is activated. Thus, the model provides for study of transcriptional regulation of the NFκB gene. NFκB is activated during inflammation, and thus the model can be used as a model of inflammation, where high luciferase activity indicated high inflammation and vice versa.

The dorsum of the mouse was shaved carefully and cleaned. SARS-CoV-2 S protein was mixed with LPS immediately before subcutaneous injection on the dorsums of the mice anesthetized with isoflurane (Baxter, Deerfield, Ill., USA). Then the animals were transferred to individually ventilated cages and imaged at 1, 3, and 6 h after the injection. An In Vivo Imaging System (IVIS Spectrum, PerkinElmer Life Sciences) was used for the longitudinal determination of NF-κB activation. Fifteen minutes before the IVIS imaging, mice were intraperitoneally given 100 μL of D-luciferin (150 mg/kg body weight). Bioluminescence from the mice was detected and quantified using Living Image 4.0 Software (PerkinElmer Life Sciences).

Mouse Pulmonary Inflammation Model

Under anesthesia, BALB/c Tg(NF-κB-RE-luc)-Xen reporter mice were instilled intratracheally with LPS (2 μg) alone, S-protein (5 μg) alone, LPS (2 μg) mixed with S-protein (5 μg), or LPS (2 μg) mixed with S-protein (5 μg) and TCP-25 (20 μg). In vivo bioimaging of NF-κB reporter gene expression was performed using the IVIS Spectrum system (PerkinElmer Life Sciences) at 6, and 24 h post intratracheal administration. Fifteen minutes before the IVIS imaging, mice were intraperitoneally given 100 μL of D-luciferin (150 mg/kg body weight). Bioluminescence from the mice was detected and quantified using Living Image 4.0 Software (PerkinElmer Life Sciences).

In another set of experiments, under anaesthesia, C57BL/6 mice were instilled intratracheally with LPS (20 μg) alone, S-protein (5 μg) alone, LPS (20 μg) mixed with S-protein (5 μg), or LPS (20 μg) mixed with S-protein (5 μg) and TCP-25 (100 μg). Bronchoalveolar lavage fluid was collected 24 h after intratracheally administration. Cytokines in the bronchoalveolar lavage fluid were measured using a mouse inflammation kit (Becton Dickinson AB) according to the manufacturer's instructions. The Mouse Inflammation Kit quantitatively measures the levels of several proteins, hereunder tumour necrosis factor-α (TNF-α), and interferon gamma (IFN- [symbol] ). The variation in the levels of these cytokines is a measure of inflammation, where high levels indicate high inflammation and vice versa, except for IL-10, where we have the opposite trend.

Neutrophil Count

Blood from the mice was collected after 24 h using cardiac puncture with syringe containing EDTA and kept on ice. Blood was then analyzed using a blood analyzer (Sysmex).

DLS

The hydrodynamic radii of E. coli LPS at increasing doses (100-500 mg/ml) alone or with S protein (1.48 mM) were measured using a DynaPro Plate reader (WYATT Technology) equipped with a temperature-controlled chamber. S protein alone was used for control. The samples were incubated in a 384-well plate at 37° C. for 30 min prior the analysis. Each measurement was performed 10 times. The hydrodynamic radii were analyzed using Dynamics 7.19 Software. The results are expressed as mean values±SD obtained from three independent experiments. In another set of experiments, 500 mg/ml LPS were incubated with different concentrations of S protein (5-250 nM) at 37° C. for 30 min. The measurements were performed as described above.

TEM Analysis

Different concentrations of E. coli LPS (100-500 mg/ml) alone or with S protein (1.48 mM) were used to prepare the samples for TEM. In brief, 5 μL of each sample were adsorbed onto carbon-coated grids (Copper mesh, 400) for 60 sec and stained with 7 μL of 2% uranyl acetate for 30 sec. The grids were rendered hydrophilic via glow discharge at low air pressure before using (Petrlova et al., 2020). Analysis was done on 15 view fields (magnification 4200×) of the mounted samples on the grid (pitch 62 μm) from three independent experiments.

Effect of SARS-CoV-2 S Protein on LPS-FITC Aggregation

LPS-FITC (5 mg/mL; Sigma) was incubated with increasing concentrations of S protein (0.0074-8880 nM) and then analyzed by recording the emission fluorescence spectra for 500-600 nm, following excitation at 488 nm. All the measurements were performed using a Jasco J-810 spectropolarimeter equipped with an FMO-427S fluorescence module, with a scan speed of 200 nm/min and 2 nm slit width. The temperature was set to 25° C. The changes in the emission of FITC-LPS as a function of change in the aggregation state of LPS endotoxin-free water was monitored at 515 nm. The experiment was performed three times.

Ethics Statement

All animal experiments are performed according to Swedish Animal Welfare Act SFS 1988:534 and were approved by the Animal Ethics Committee of Malm6/Lund, Sweden.

Statistical Analysis

All in vitro assays were repeated at least three times. Unless otherwise stated. Data are presented as means±SEM. Differences in the mean between two groups were analyzed using Student's t test for normally distributed data and Mann-Whitney test otherwise. To compare means between more than two groups, a one-way ANOVA with Dunnet or Holm-Sidak posttest were used. Statistical analysis, as indicated in each figure legend, were performed using GraphPad Prism software v8. P values<0.05 were considered to be statistically significant.

### REFERENCES

A. Shevchenko, H. Tomas, J. Havlis, J. V. Olsen, M. Mann, In-gel digestion for mass spectrometric characterization of proteins and proteomes. Nat Protoc 1, 2856-2860 (2006).

1. A compound for use in a method of treatment of infection by S protein virus or treatment of inflammation associated with infection by S protein virus in an individual in need thereof, wherein the compound comprises a peptide comprising or consisting of the amino acid sequence
X₁-X₂-X₃-X₄-X₅-X₆-W-X₈-X₉-X₁₀, wherein
X_{4, 6, 9} is any standard amino acid,
X₁ is I, L or V,
X₂ is any standard amino acid except C,
X₃ is A, E, Q, R or Y,
X₅ is any standard amino acid except R,
X₈ is I or L,
X₁₀ is any standard amino acid except H,
wherein said peptide has a length of from 10 to 100 amino acid residues.

2. The compound for use according to claim 1, wherein the virus is selected from the group consisting of viruses from the Coronaviridae family, preferably wherein the virus is selected from the group of viruses disclosed in FIGS. 9 and 10, more preferably wherein the virus is selected from the group consisting of PorCov-HKU15, SARS-CoV, HCoV NL63, HKU1, MERS-CoV, SARS-CoV 2, and MERS-CoV, most preferably wherein the virus is SARS-CoV 2.

3. The compound for use according to any one of the preceding claims, wherein the inflammation is a local inflammatory disease associated with said infection by virus, optionally wherein the inflammation is an inflammatory disease selected from the group consisting of acute respiratory distress syndrome (ARDS), severe acute respiratory syndrome (SARS), gastroenteritis and upper and/or lower respiratory tract infections, for example Pneumonia.

4. The compound for use according to any one of the preceding claims, wherein said individual further suffers from a bacterial infection, such as an acute or chronic bacterial infection, for example infection by gram negative bacteria.

5. The compound for use according to any one of the preceding claims, wherein the individual suffers from an S protein virus infection and a bacterial infection of the lungs, optionally wherein said individual has an increased level of LPS in bronchoalveolar lavage.

6. The compound for use according to any one of the preceding claims, wherein said individual has increased level of LPS in one or more body fluids, optionally wherein said body fluid is selected from the group consisting of blood, serum, saliva, nasopharyngeal swab samples and bronchoalveolar lavage (BAL) samples.

7. The compound for use according to any one of claims 5 to 6, wherein said increased level of LPS is a level of at least 50 pg/ml.

8. The compound for use according to any one of the preceding claims, wherein the individual is an animal, such as a domestic animal, for example an animal selected from the group consisting of pig, cattle, cat, poultry, dog and mink, and wherein the compound is for use in treatment of porcine respiratory coronavirus infection, shipping fever induced by bovine coronavirus, feline CoV or infectious bronchitis virus.

9. The compound for use according to any one of the preceding claims, wherein said compound is for use in a method of blocking inflammation induced by Spike protein.

10. The compound for use according to any one of the preceding claims, wherein said S protein is selected from the group consisting of SARS CoV-2 S protein of SEQ ID NO:8 and related S proteins having at least 20%, such as at least 25% sequence identity therewith, optionally wherein said S protein is selected from the group consisting of S protein of SEQ ID NO: 8, SEQ ID NO: 9, SEQ ID NO: 10, SEQ ID NO: 11, SEQ ID NO: 12, SEQ ID NO: 13 and S proteins sharing 95% sequence identity to one of the aforementioned, further optionally wherein said S protein is any of the S proteins mentioned in FIGS. 9 and 10.

11. A method for forecasting the outcome of infection by S protein virus in an individual suffering from or at risk of acquiring infection by said virus, said method comprising the steps of
a. providing a sample from said individual
b. determining lipopolysaccharide (LPS) level in said samples
wherein increased LPS levels are indicative of a severe outcome.

12. The method according to claim 11, wherein the method is a method for predicting the risk of ARDS, wherein high level of LPS is indicative of risk of ARDS, optionally wherein said method further comprises administering a compound comprising a peptide as defined in claim 1 to said individual if said LPS levels are increased.

13. The method according to any one of claims 11 to 12, wherein said sample is selected from the group consisting of blood, serum, saliva, nasopharyngeal swab samples and bronchoalveolar lavage (BAL) samples.

14. The method according to any one of claims 11 to 13, wherein said increased level of LPS is a level of at least 50 pg/ml, such as wherein said increased level of LPS is a serum level of LPS of at least 50 pg/ml.

15. The method according to any one of claims 11 to 14, wherein said S protein virus is as defined in claim 2.

16. The compound for use according to any one of the preceding claims, wherein
i) the peptide comprises or consists of the amino acid sequence
X1-X2-X3-X4-X5-X6-W-X8-X9-X10-X11-X12-X13, wherein
X4, 6, 9, 11 is any standard amino acid,
X1, is I, L or V
X2 is any standard amino acid except C
X3 is A, E, Q, R or Y
X5 is any standard amino acid except R
X8 is I or L
X10 is any standard amino acid except H
X12 is I, M or T
X13 is D, K, Q or R
and wherein said peptide has a length of from 20 to 100 amino acid residues; or
ii) the peptide comprises or consists of the amino acid sequence
X1-X2-X3-X4-X5-X6-W-X8-X9-X10-X11-X12-X13-X14-X15-X16-X17,
wherein
X4, 6, 9, 11, 14, 15 is any standard amino acid
X1 is I, L or V
X2 is any standard amino acid except C
X3 is A, E, Q, R or Y
X5 is any standard amino acid except R
X8 is I or L
X10 is any standard amino acid except H
X12 is I, M or T
X13 is D, K, Q or R
X16 is G or D
X17 is E, L, G, R or K
and wherein said peptide has a length of from 20 to 100 amino acid residues.

17. The compound for use according to any one of the preceding claims, wherein the peptide has a length of 18 to 35 amino acids, preferably 18-25 amino acids, and comprises or consists of any of the amino acid sequences
(SEQ ID NO: 1)
GKYGFYTHVFRLKKWIQKVIDQFGE,
(SEQ ID NO: 2)
FYTHVFRLKKWIQKVIDQFGE,
(SEQ ID NO: 3)
GKYGFYTHVFRLKKWIQKVI,
(SEQ ID NO: 4)
HVFRLKKWIQKVIDQFGE,
(SEQ ID NO: 5)
KYGFYTHVFRLKKWIQKVIDQFGE
(SEQ ID NO: 6)
GKYGFYTHVFRLKKWIQKVIDQF
or
(SEQ ID NO: 7)
GKYGFYTHVFRLKKWIQKV.
preferably wherein the peptide comprises or consists of any of the amino acid sequences
(SEQ ID NO: 1)
GKYGFYTHVFRLKKWIQKVIDQFGE,
or
(SEQ ID NO: 3)
GKYGFYTHVFRLKKWIQKVI.
preferably wherein the peptide has at least 90% sequence identity with the amino acid sequence GKYGFYTHVFRLKKWIQKVIDQFGE (SEQ ID NO: 1);
preferably wherein the peptide consists of the amino acid sequence
(SEQ ID NO: 1)
GKYGFYTHVFRLKKWIQKVIDQFGE.

18. The compound for use according to any one of the preceding claims, wherein one or more of the standard amino acids comprised in the TCP peptide are modified or derivatised, optionally wherein one or more of the standard amino acids comprised in the TCP peptide are PEGylated, amidated, acylated, acetylated, alkenylated and/or alkylated.


SEQUENCE LISTING (13 sequences)

SEQ ID NO: 1 - protein, 25 aa, Artificial sequence
  Peptide derived from human thrombin
GKYGFYTHVFRLKKWIQKVIDQFGE

SEQ ID NO: 2 - protein, 21 aa, Artificial sequence
  Peptide derived from human thrombin
FYTHVFRLKKWIQKVIDQFGE

SEQ ID NO: 3 - protein, 20 aa, Artificial sequence
  Peptide derived from human thrombin
GKYGFYTHVFRLKKWIQKVI

SEQ ID NO: 4 - protein, 18 aa, Artificial sequence
  Peptide derived from human thrombin
HVFRLKKWIQKVIDQFGE

SEQ ID NO: 5 - protein, 24 aa, Artificial sequence
  Peptide derived from human thrombin
KYGFYTHVFRLKKWIQKVIDQFGE

SEQ ID NO: 6 - protein, 23 aa, Artificial sequence
  Peptide derived from human thrombin
GKYGFYTHVFRLKKWIQKVIDQF

SEQ ID NO: 7 - protein, 19 aa, Artificial sequence
  Peptide derived from human thrombin
GKYGFYTHVFRLKKWIQKV

SEQ ID NO: 8 - protein, 1273 aa, SARS-Cov-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 9 - protein, 1255 aa, SARS-CoV
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 10 - protein, 1351 aa, HCoV-HKU1
MFLIIFILPTTLAVIGDFNCTNSFINDYNKTIPRISEDVVDVSLGLGTYYVLNRVYLNTTLLFTGYFPKSGANFRDLALKGSIYLSTLWYKPPFLSDFNNGIFSKVKNTKLYVNNTLYSEFSTIVIGSVFVNTSYTIVVQPHNGILEITACQYTMCEYPHTVCKSKGSIRNESWHIDSSEPLCLFKKNFTYNVSADWLYFHFYQERGVFYAYYADVGMPTTFLFSLYLGTILSHYYVMPLTCNAISSNTDNETLEYWVTPLSRRQYLLNFDEHGVITNAVDCSSSFLSEIQCKTQSFAPNTGVYDLSGFTVKPVATVYRRIPNLPDCDIDNWLNNVSVPSPLNWERRIFSNCNFNLSTLLRLVHVDSFSCNNLDKSKIFGSCFNSITVDKFAIPNRRRDDLQLGSSGFLQSSNYKIDISSSSCQLYYSLPLVNVTINNFNPSSWNRRYGFGSFNLSSYDVVYSDHCFSVNSDFCPCADPSVVNSCAKSKPPSAICPAGTKYRHCDLDTTLYVKNWCRCSCLPDPISTYSPNTCPQKKVVVGIGEHCPGLGINEEKCGTQLNHSSCFCSPDAFLGWSFDSCISNNRCNIFSNFIFNGINSGTTCSNDLLYSNTEISTGVCVNYDLYGITGQGIFKEVSAAYYNNWQNLLYDSNGNIIGFKDFLTNKTYTILPCYSGRVSAAFYQNSSSPALLYRNLKCSYVLNNISFISQPFYFDSYLGCVLNAVNLTSYSVSSCDLRMGSGFCIDYALPSSRRKRRGISSPYRFVTFEPFNVSFVNDSVETVGGLFEIQIPTNFTIAGHEEFIQTSSPKVTIDCSAFVCSNYAACHDLLSEYGTFCDNINSILNEVNDLLDITQLQVANALMQGVTLSSNLNTNLHSDVDNIDFKSLLGCLGSQCGSSSRSLLEDLLFNKVKLSDVGFVEAYNNCTGGSEIRDLLCVQSFNGIKVLPPILSETQISGYTTAATVAAMFPPWSAAAGVPFSLNVQYRINGLGVTMDVLNKNQKLIANAFNKALLSIQNGFTATNSALAKIQSVVNANAQALNSLLQQLFNKFGAISSSLQEILSRLDNLEAQVQIDRLINGRLTALNAYVSQQLSDITLIKAGASRAIEKVNECVKSQSPRINFCGNGNHILSLVQNAPYGLLFIHFSYKPTSFKTVLVSPGLCLSGDRGIAPKQGYFIKQNDSWMFTGSSYYYPEPISDKNVVFMNSCSVNFTKAPFIYLNNSIPNLSDFEAELSLWFKNHTSIAPNLTFNSHINATFLDLYYEMNVIQESIKSLNSSFINLKEIGTYEMYVKWPWYIWLLIVILFIIFLMILFFICCCTGCGSACFSKCHNCCDEYGGHNDFVIKASHDD

SEQ ID NO: 11 - protein, 1363 aa, Bovine coronavirus
MFLILLISLPMAFAVIGDLKCTTVSINDVDTGAPSISTDIVDVTNGLGTYYVLDRVYLNTTLLLNGYYPTSGSTYRNMALKGTLLLSRLWFKPPFLSDFINGIFAKVKNTKVIKKGVMYSEFPAITIGSTFVNTSYSVVVQPHTTNLDNKLQGLLEISVCQYTMCEYPHTICHPNLGNKRVELWHWDTGVVSCLYKRNFTYDVNADYLYFHFYQEGGTFYAYFTDTGVVTKFLFNVYLGTVLSHYYVLPLTCSSAMTLEYWVTPLTSKQYLLAFNQDGVIFNAVDCKSDFMSEIKCKTLSIAPSTGVYELNGYTVQPIADVYRRIPNLPDCNIEAWLNDKSVPSPLNWERKTFSNCNFNMSSLMSFIQADSFTCNNIDAAKIYGMCFSSITIDKFAIPNGRKVDLQLGNLGYLQSFNYRIDTTATSCQLYYNLPAANVSVSRFNPSTWNRRFGFTEQFVFKPQPVGVFTHHDVVYAQHCFKAPSNFCPCKLDGSLCVGNGPGIDAGYKNSGIGTCPAGTNYLTCHNAAQCNCLCTPDPITSKSTGPYKCPQTKYLVGIGEHCSGLAIKSDYCGGNPCTCQPQAFLGWSVDSCLQGDRCNIFANFILHDVNSGTTCSTDLQKSNTDIILGVCVNYDLYGITGQGIFVEVNATYYNSWQNLLYDSNGNLYGFRDYLTNRTFMIRSCYSGRVSAAFHANSSEPALLFRNIKCNYVFNNTLSRQLQPINYFDSYLGCVVNADNSTSSVVQTCDLTVGSGYCVDYSTKRRSRRAITTGYRFTTFEPFTVNSVNDSLEPVGGLYEIQIPSEFTIGNMEEFIQTSSPKVTIDCSAFVCGDYAACKSQLVEYGSFCDNINAILTEVNELLDTTQLQVANSLMNGVTLSTKLKDGVNFNVDDINFSPVLGCLGSDCNKVSSRSAIEDLLFSKVKLSDVGFVEAYNNCTGGAEIRDLICVQSYNGIKVLPPLLSVNQISGYTLAATSASLFPPLSAAVGVPFYLNVQYRINGIGVTMDVLSQNQKLIANAFNNALDAIQEGFDATNSALVKIQAVVNANAEALNNLLQQLSNRFGAISSSLQEILSRLDALEAQAQIDRLINGRLTALNVYVSQQLSDSTLVKFSAAQAMEKVNECVKSQSSRINFCGNGNHIISLVQNAPYGLYFIHFSYVPTKYVTAKVSPGLCIAGDRGIAPKSGYFVNVNNTWMFTGSGYYYPEPITGNNVVVMSTCAVNYTKAPDVMLNISTPNLHDFKEELDQWFKNQTSVAPDLSLDYINVTFLDLQDEMNRLQEAIKVLNQSYINLKDIGTYEYYVKWPWYVWLLIGFAGVAMLVLLFFICCCTGCGTSCFKICGGCCDDYTGHQELVIKTSHDD

SEQ ID NO: 12 - protein, 1353 aa, MERS-CoV S
MIHSVFLLMFLLTPTESYVDVGPDSVKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYESFDVESGVYSVSSFEAKPSGSVVEQAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVPQLVNANQYSPCVSIVPSTVWEDGDYYRKQLSPLEGGGWLVASGSTVAMTEQLQMGFGITVQYGTDTNSVCPKLEFANDTKIASQLGNCVEYSLYGVSGRGVFQNCTAVGVRQQRFVYDAYQNLVGYYSDDGNYYCLRACVSVPVSVIYDKETKTHATLFGSVACEHISSTMSQYSRSTRSMLKRRDSTYGPLQTPVGCVLGLVNSSLFVEDCKLPLGQSLCALPDTPSTLTPRSVRSVPGEMRLASIAFNHPIQVDQLNSSYFKLSIPTNFSFGVTQEYIQTTIQKVTVDCKQYVCNGFQKCEQLLREYGQFCSKINQALHGANLRQDDSVRNLFASVKSSQSSPIIPGFGGDFNLTLLEPVSISTGSRSARSAIEDLLFDKVTIADPGYMQGYDDCMQQGPASARDLICAQYVAGYKVLPPLMDVNMEAAYTSSLLGSIAGVGWTAGLSSFAAIPFAQSIFYRLNGVGITQQVLSENQKLIANKFNQALGAMQTGFTTTNEAFRKVQDAVNNNAQALSKLASELSNTFGAISASIGDIIQRLDVLEQDAQIDRLINGRLTTLNAFVAQQLVRSESAALSAQLAKDKVNECVKAQSKRSGFCGQGTHIVSFVVNAPNGLYFMHVGYYPSNHIEVVSAYGLCDAANPTNCIAPVNGYFIKTNNTRIVDEWSYTGSSFYAPEPITSLNTKYVAPHVTYQNISTNLPPPLLGNSTGIDFQDELDEFFKNVSTSIPNFGSLTQINTTLLDLTYEMLSLQQVVKALNESYIDLKELGNYTYYNKWPWYIWLGFIAGLVALALCVFFILCCTGCGTNCMGKLKCNRCCDRYEEYDLEPHKVHVH

SEQ ID NO: 13 - protein, 1225 aa, Porcine respiratory coronovirus
MKKLFVVLVVMPLIYGDKFPTSVVSNCTDQCASYVANVFTTQPGGFIPSDFSFNNWFLLTNSSTLVSGKLVTKQPLLVNCLWPVPSFEEAASTFCFEGADFDQCNGAVLNNTVDVIRFNLNFTTNVQSGKGATVFSLNTTGGVTLEISCYNDTVSDSSFSSYGEIPFGVTNGPRYCYVLYNGTALKYLGTLPPSVKEIAISKWGHFYINGYNFFSTFPIDCISFNLTTGDSDVFWTIAYTSYTEALVQVENTAITNVTYCNSYVNNIKCSQLTANLNNGFYPVSSSEVGSVNKSVVLLPSFLTHTIVNITIGLGMKRSGYGQPIASTLSNITLPMQDNNTDVYCVRSDQFSVYVHSTCKSALWDNVFKRNCTDVLDATAVIKTGTCPFSFDKLNNYLTFNKFCLSLSPVGANCKFDVAARTRTNEQVVRSLYVIYEEGDSIVGVPSDNSGLHDLSVLHLDSCTDYNIYGRTGVGIIRQTNRTLLSGLYYTSLSGDLLGFKNVSDGVIYSVTPCDVSAQAAVIDGTIVGAITSINSELLGLTHWTITPNFYYYSIYNYTNDKTRGTPIDSNDVGCEPVITYSNIGVCKNGALVFINVTHSDGDVQPISTGNVTIPTNFTISVQVEYIQVYTTPVSIDCSRYVCNGNPRCNKLLTQYVSACQTIEQALAMGARLENMEVDSMLFVSENALKLASVEAFNSSETLDPIYTQWPNIGGFWLEGLKYILPSDNSKRKYRSAIEDLLFSKVVTSGLGTVDEDYKRCTGGYDIADLVCAQYYNGIMVLPGVANADKMTMYTASLAGGITLGAFGGGAVSIPFAVAVQARLNYVALQTDVLNKNQQILASAFNQAIGNITQSFGKVNDAIHQTSRGLTTVAKALAKVQDVVNTQGQALRHLTVQLQNNFQAISSSISDIYNRLDELSADAQVDRLITGRLTALNAFVSQTLTRQAEVRASRQLAKDKVNECVRSQSQRFGFCGNGTHLFSLANAAPNGMIFFHTVLLPTAYETVTAWSGICALDGDRTFGLVVKDVQLTLFRNLDDKFYLTPRTMYQPRVATSSDFVQIEGCDVLFVNTTVSDLPSIIPDYIDINQTVQDILENFRPNWTVPELTLDVFNATYLNLTGEIDDLEFRSEKLHNTTVELAILIDNINNTLVNLEWLNRIETYVKWPWYVWLLIGLVVIFCIPLLLFCCCSTGCCGCIGCLGSCCHSIFSRRQFENYEPIEKVHVH